(19) United States
(12) Patent Application Publication
(10) Pub. No.: US 2024/0400491 A1
(43) Pub. Date: Dec. 5, 2024

(54) NEW HETEROGENEOUS PALLADIUM-BASED CATALYST, PREPARATION METHOD AND USE THEREOF

(71) Applicant: FAIRBRICS, CLICHY (FR)

(72) Inventors: Ibrahim ABDELLAH, Montigny Les Metz (FR); Bidal YANNICK, POIGNY LA FORÊT (FR); Tawfiq NASR ALLAH, Paris (FR)

(73) Assignee: FAIRBRICS, CLICHY (FR)

(21) Appl. No.: 18/690,809

(22) PCT Filed: Sep. 9, 2022
(86) PCT No.: PCT/EP2022/075148

§ 371 Date: Mar. 11, 2024

(30) Foreign Application Priority Data
Sep. 10, 2021 (FR) FR2109520

(51) Int. Cl. C07C 67/36; B01J 23/63; B01J 35/61; B01J 37/02; B01J 37/08

(52) U.S. Cl. CPC C07C 67/36; B01J 23/63; B01J 35/615; B01J 37/0201; B01J 37/08

(57) ABSTRACT

A new catalyst that includes palladium on a cerium dioxide support, of formula Pd—X/CeO2, in which X represents the empty set or a doping element, and its use in the implementation of a method for selectively preparing oxalates or oxamides from carbon monoxide, an oxidant, in particular molecular oxygen or air, and an alcohol or an amine respectively.

This invention concerns a new heterogeneous palladium-based catalyst, its method of preparation and its use in the synthesis of oxalates and oxamides.

### CONTEXT OF THE INVENTION

Oxalates and oxamides are high value-added molecules in many areas of the chemical industry, with a wide range of applications. They are particularly interesting as precursors for other molecules of interest, such as ethylene glycol. Their production by catalytic means, by palladium (Pd) catalysts, is a method implemented in the prior art.

Multiple synthesis routes have been described:
using nitrites or nitrates, for example,
non-recyclable homogeneous catalysts,
Pd catalysts on coal, which present safety risks,
Pd catalysts on different supports.

Gaffney et al (Journal of Catalysis, 1984, 90, 261-269) studied various oxides as supports for the heterogeneous catalysis of Pd in an oxidative carbonylation reaction at a high pressure of over 17 MPa. According to their study, some of these oxides act as co-oxidants. In particular, the authors focused on a Pd catalyst supported on a mixture of vanadium and titanium oxides. Today, a heterogeneous catalyst is sought for the versatile synthesis of oxalates and oxamides that is environmentally friendly and can be industrialised in terms of simplicity, efficiency and safety.

One of the aims of the invention is to propose the use of a new heterogeneous palladium catalyst which enables oxalate or oxamide compounds to be prepared.

One of the aims of the invention is to propose the use of a new heterogeneous palladium catalyst which enables oxalate or oxamide compounds to be prepared from carbon monoxide (CO), an oxidant, in particular molecular oxygen (O₂) or air, and an alcohol or an amine respectively. Another aim of the invention is to prepare oxalates or oxamides without using toxic and explosive reagents such as nitro derivatives.

Another aim of the invention is the preparation of environmentally-friendly oxalates and oxamides.

Another aim of the invention is the preparation of oxalates and oxamides using recyclable reagents.

Another aim of the invention is to provide a heterogeneous palladium catalyst that is efficient, reusable and recyclable.

Another aim of the invention is to provide a heterogeneous palladium catalyst for use in a continuous flow process.

Another aim of the present invention is to propose a simple and optimised method for preparing this palladium catalyst.

A first object of the present invention is the use of a palladium/cerium dioxide catalyst (Pd/CeO₂), comprising palladium on a cerium dioxide support, in the implementation of a method for selectively preparing oxalates or oxamides from carbon monoxide (CO), an oxidant, in particular molecular oxygen (O₂) or air, and an alcohol or an amine respectively.

Within the meaning of this invention, “palladium/cerium dioxide catalyst” or “Pd/CeO₂” means a catalyst in which the palladium atoms are catalytic sites bonded to a cerium dioxide support. It is understood that the catalyst may include other elements such as dopants.

According to a particular embodiment, the invention relates to the use as defined above, said catalyst being of formula Pd—X/CeO₂, in which X represents the empty set or a doping element. When X represents the empty set, the catalyst consists of palladium on a cerium dioxide support.

Where X represents an atomic element, the catalyst is doped with element X and comprises palladium and element X on a cerium dioxide support.

The use according to the invention can combine the following 3 features:
using a catalyst with a specific surface area from 50 to 250 m²/g,
the presence of a promoter and
implementation at a pressure from 0.1 to 15 MPa.

According to a particular embodiment, the invention relates to the use of a Pd—X/CeO₂ catalyst comprising palladium on a cerium dioxide support, in which the catalyst has a surface area, analysed by BET, from 50 to 250 m²/g, in particular from 100 to 200 m²/g, in the implementation of a method for selectively preparing oxalates or oxamides from carbon monoxide, an oxidant and an alcohol or an amine respectively.

The expression “from 50 to 250 m²/g” corresponds to the ranges: from 50 to 60 m²/g; from 60 to 70 m²/g; from 70 to 80 m²/g; from 80 to 90 m²/g; from 90 to 100 m²/g; from 100 to 110 m²/g; from 110 to 120 m²/g; from 120 to 130 m²/g; from 130 to 140 m²/g; from 140 to 150 m²/g; from 150 to 160 m²/g; from 160 to 170 m²/g; from 170 to 180 m²/g; from 180 to 190 m²/g; from 190 to 200 m²/g; from 200 to 210 m²/g; from 210 to 220 m²/g; from 220 to 230 m²/g; from 230 to 240 m²/g; from 240 to 250 m²/g.

The expression 100 to 200 m²/g corresponds to the ranges from 100 to 110 m²/g; from 110 to 120 m²/g; from 120 to 130 m²/g; from 130 to 140 m²/g; from 140 to 150 m²/g; from 150 to 160 m²/g; from 160 to 170 m²/g; from 180 to 190 m²/g; from 190 to 200 m²/g.

According to a particular embodiment, the invention relates to the use of a Pd—X/CeO₂ catalyst comprising palladium on a cerium dioxide support, in which the catalyst has a surface area, analysed by BET, from 100 to 250 m²/g, for the implementation of a method for selectively preparing oxalates or oxamides from carbon monoxide, an oxidant and an alcohol or an amine respectively.

According to a particular embodiment, the invention relates to the use of a Pd—X/CeO₂ catalyst comprising palladium on a cerium dioxide support, in which the catalyst has a surface area, analysed by BET, from 100 to 200 m²/g, for the implementation of a method for selectively preparing oxalates or oxamides from carbon monoxide, an oxidant and an alcohol or an amine respectively.

The inventors have unexpectedly observed that a catalyst the surface area of which, analysed by BET, is from 100 and 250 m²/g, in particular from 100 and 200 m²/g, exhibits higher yields than a palladium catalyst on cerium oxide according to the prior art, in particular according to Gaffney et al (Journal of Catalysis 90, 261-269, 1984) as shown below (see example 10).

Furthermore, two Pd—X/CeO₂ catalysts according to the invention with surface areas in this range of 100 to 250 m²/g have substantially equivalent activities, as shown below (see example 8). It would rather be expected that a catalyst with a larger specific surface area would be more favourable in terms of catalysis because it would promote exchanges.

According to a particular embodiment, the invention concerns the use of a Pd—X/CeO₂ catalyst, comprising palladium on a cerium dioxide support, in the implementation of a method for selectively preparing oxalates or oxamides from carbon monoxide (CO), an oxidant and an alcohol or an amine respectively, in the presence of a promoter.

According to one embodiment and in the following, the promoter is a reaction promoter.

According to a particular embodiment, the invention concerns the use of a Pd—X/CeO₂ catalyst, comprising palladium on a cerium dioxide support, in the implementation of a method for selectively preparing oxalates or oxamides from carbon monoxide (CO), an oxidant and an alcohol or an amine respectively, at a pressure of 0.1 to 15 MPa.

The expression MPa corresponds to 106 Pascal and is equivalent to 10 bars.

The expression “from 0.1 to 15.0 MPa” corresponds to the ranges: 0.1 to 0.5 MPa; 0.5 to 1.0 MPa; 1.0 to 1.5 MPa; 1.5 to 2.0 MPa; 2.0 to 2.5 MPa; 2.5 to 3.0 MPa; 3.0 to 3.5 MPa; 3.5 to 4.0 MPa; 4.0 to 4.5 MPa; 4.5 to 5.0 MPa; 5.0 to 5.5 MPa; 5.5 to 6.0 MPa; 6.0 to 6.5 MPa; 6.5 to 7.0 MPa; 7.0 to 7.5 MPa; 7.5 to 8.0 MPa; 8.0 to 8.5 MPa; 8.5 to 9.0 MPa; 9.0 to 9.5 MPa; 9.5 to 10.0 MPa; 10.0 to 10.5 MPa; 10.5 to 11.0 MPa; 11.0 to 11.5 MPa; 11.5 to 12.0 MPa; 12.0 to 12.5 MPa; 12.5 to 13.0 MPa; 13.0 to 13.5 MPa; 13.5 to 14.0 MPa; 14.0 to 14.5 MPa; 14.5 to 15.0 MPa.

According to a particular embodiment, the invention relates to the use of a Pd—X/CeO₂ catalyst, comprising palladium on a cerium dioxide support, in which the catalyst has a surface area, analysed by BET, from 50 and 250 m²/g, in particular from 100 to 250 m²/g, in particular from 100 to 200 m²/g, in the implementation of a method for selectively preparing oxalates or oxamides, from carbon monoxide, an oxidant and an alcohol or an amine respectively, in the presence of a promoter.

According to a particular embodiment, the invention relates to the use of a Pd—X/CeO₂ catalyst, comprising palladium on a cerium dioxide support, in which the catalyst has a surface area, analysed by BET, from 50 to 250 m²/g, in particular from 100 to 250 m²/g, in particular from 100 to 200 m²/g, in the implementation of a method for selectively preparing oxalates or oxamides, from carbon monoxide, an oxidant and an alcohol or an amine respectively, at a pressure of from 0.1 to 15 MPa.

According to a particular embodiment, the invention concerns the use of a Pd—X/CeO₂ catalyst, comprising palladium on a cerium dioxide support, in the implementation of a method for selectively preparing oxalates or oxamides from carbon monoxide (CO), an oxidant and an alcohol or an amine respectively, in the presence of a promoter and at a pressure of 0.1 to 15 MPa.

According to a particular embodiment, the invention relates to the use of a Pd—X/CeO₂ catalyst, comprising palladium on a cerium dioxide support, in which the catalyst has a surface area, analysed by BET, from 50 and 250 m²/g, in particular from 100 to 250 m²/g, in particular from 100 to 200 m²/g, in the implementation of a method for selectively preparing oxalates or oxamides, from carbon monoxide, an oxidant and an alcohol or an amine respectively, in the presence of a promoter and at a pressure of from 0.1 to 15 MPa.

According to a particular embodiment, the invention relates to the use as defined above in which the oxidant is chosen from: molecular oxygen (O₂), air, a dione, in particular 1,4-benzoquinone, 1,4-dicloro-2-butene and CuCl₂.

For the purposes of this invention, air is defined as an oxidant. Air is a gas composition comprising, in molar fraction, approximately 78% nitrogen (N₂), 21% oxygen O₂ and approximately less than 1% of other gases including carbon dioxide (CO₂), methane (CH₄) and rare gases including argon, helium, neon, krypton and xenon. Nitrogen is an inert gas, so the oxidising reactivity of air is governed by that of oxygen. Dioxygen is also called molecular oxygen or oxygen in the present invention.

Advantageously, the invention relates to the use as defined above in which molecular oxygen (O₂) or air is used as the oxidant.

According to a particular embodiment, the invention relates to the use of a Pd—X/CeO₂ catalyst, comprising palladium on a cerium dioxide support, in which the catalyst has a surface area, analysed by BET, from 50 to 250 m²/g, in particular from 100 to 250 m²/g, in particular from 100 to 200 m²/g, in the implementation of a method for selectively preparing oxalates or oxamides from carbon monoxide, molecular oxygen or air and an alcohol or an amine respectively.

According to a particular embodiment, the invention concerns the use of a Pd—X/CeO₂ catalyst, comprising palladium on a cerium dioxide support, in the implementation of a method for selectively preparing oxalates or oxamides from carbon monoxide (CO), molecular oxygen (O₂) or air, and an alcohol or an amine respectively, in the presence of a promoter.

According to a particular embodiment, the invention relates to the use of a Pd—X/CeO₂ catalyst, comprising palladium on a cerium dioxide support, in the implementation of a method for selectively preparing oxalates or oxamides from carbon monoxide (CO), molecular oxygen (O₂) or air, and an alcohol or an amine respectively, at a pressure of 0.1 to 15 MPa.

According to a particular embodiment, the invention relates to the use of a Pd—X/CeO₂ catalyst, comprising palladium on a cerium dioxide support, in which the catalyst has a surface area, analysed by BET, from 50 to 250 m²/g, in particular from 100 to 250 m²/g, in particular from 100 to 200 m²/g, in the implementation of a method for selectively preparing oxalates or oxamides from carbon monoxide, molecular oxygen or air and an alcohol or an amine respectively, in the presence of a promoter.

According to a particular embodiment, the invention relates to the use of a Pd—X/CeO₂ catalyst, comprising palladium on a cerium dioxide support, in which the catalyst has a surface area, analysed by BET, from 50 to 250 m²/g, in particular from 100 to 250 m²/g, in particular from 100 to 200 m²/g, in a method for selectively preparing oxalates or oxamides from carbon monoxide, molecular oxygen or air and an alcohol or an amine respectively, at a pressure of from 0.1 to 15 MPa.

According to a particular embodiment, the invention relates to the use of a Pd—X/CeO₂ catalyst, comprising palladium on a cerium dioxide support, in the implementation of a method for selectively preparing oxalates or oxamides, from carbon monoxide (CO), molecular oxygen (O₂) or air, and an alcohol or an amine respectively, in the presence of a promoter and at a pressure of 0.1 to 15 MPa.

According to a particular embodiment, the invention relates to the use of a Pd—X/CeO₂ catalyst, comprising palladium on a cerium dioxide support, in which the catalyst has a surface area, analysed by BET, from 50 to 250 m²/g, in particular from 100 to 250 m²/g, in particular from 100 to 200 m²/g, in a method for selectively preparing oxalates or oxamides from carbon monoxide, molecular oxygen or air and an alcohol or an amine respectively, in the presence of a promoter and at a pressure of from 0.1 to 15 MPa.

According to a particular embodiment, the invention relates to the use of a palladium/cerium dioxide catalyst, comprising palladium on a cerium dioxide support, of formula Pd—X/CeO₂, in which X represents the empty set or a doping element, in which the catalyst has a surface area, analysed by BET, from 50 to 250 m²/g, in particular from 100 to 250 m²/g, in particular from 100 to 200 m²/g, in the implementation of a method for selectively preparing oxalates or oxamides from carbon monoxide, an oxidant, in particular molecular oxygen or air, and an alcohol or an amine respectively, optionally in the presence of a promoter and optionally at a pressure of from 0.1 to 15 MPa.

By “oxalate” it is meant the dialkyloxalate corresponding to the alcohol used.

By “oxamide” it is meant the 1,1′-oxalyl diamine oxamide derivative corresponding to the amine used.

It is understood that the Pd—X/CeO₂ catalyst of the invention is a heterogeneous catalyst. The advantage of using a heterogeneous catalyst is that it is easier to separate the catalyst from the other species involved in the reaction, making it easy to recover and reuse the catalyst. The use of a heterogeneous catalyst also has the advantage of making it possible to fix the catalyst in the reactor in an enclosure such as a cartridge when operating under continuous flow, and thus to obtain catalyst-free products at the reactor outlet.

By “surface area” it is meant the surface area accessible to gases and liquids. It is measured in m²/g using well-known techniques such as the BET method (Brunauer, Emmett and Teller).

By “promoter” or “reaction promoter” it is meant a substance which can improve the properties of a catalyst, such as catalytic activity, selectivity, anti-toxicity, stability, lifetime or prevent deactivation of the catalyst.

In a particular embodiment, the promoter is an introduced salt or an introduced molecular species. It is understood that in this particular embodiment the promoter is not bound or comprised in the support.

In a particular embodiment, the promoter is an oxidant. The promoter can thus promote the oxidative carbonylation method.

According to a particular embodiment, the invention relates to the use as defined above, the implementation of the method comprising at least one additive.

By “additive” it is meant a substance that improves the yield of the reaction but is not essential to its progress.

In a particular embodiment, the additive is a base.

The expression “a method for selectively preparing product” refers to a method enabling to obtain the desired product, the oxalate or the oxamide, with a selectivity of more than 50%.

The invention can be used independently to prepare oxalates or oxamides.

According to a particular embodiment, the invention relates to the use, as defined above, of a Pd—X/CeO₂ catalyst, comprising palladium on a cerium dioxide support, in the implementation of a method for selectively preparing oxalates from carbon monoxide (CO), an oxidant and an alcohol.

According to a particular embodiment, the invention relates to the use, as defined above, of a Pd—X/CeO₂ catalyst, comprising palladium on a cerium dioxide support, in the implementation of a method for selectively preparing oxamides from carbon monoxide (CO), an oxidant and an amine.

According to a particular embodiment, the invention relates to the use as defined above of a Pd—X/CeO₂ catalyst, comprising palladium on a cerium dioxide support, in which the catalyst has a surface area, analysed by BET, from 50 to 250 m²/g, in particular from 100 to 250 m²/g, in particular from 100 to 200 m²/g, in the implementation of a method for selectively preparing oxalates from carbon monoxide, an oxidant and an alcohol.

According to a particular embodiment, the invention relates to the use as defined above of a Pd—X/CeO₂ catalyst, comprising palladium on a cerium dioxide support, in which the catalyst has a surface area, analysed by BET, from 50 to 250 m²/g, in particular from 100 to 250 m²/g, in particular from 100 to 200 m²/g, in the implementation of a method for selectively preparing oxamides from carbon monoxide, an oxidant and an amine.

According to a particular embodiment, the invention relates to the use, as defined above, of a Pd—X/CeO₂ catalyst, comprising palladium on a cerium dioxide support, in the implementation of a method for selectively preparing oxalates, from carbon monoxide (CO), an oxidant and an alcohol, in the presence of a promoter.

According to a particular embodiment, the invention relates to the use, as defined above, of a Pd—X/CeO₂ catalyst, comprising palladium on a cerium dioxide support, in the implementation of a method for selectively preparing oxamides, from carbon monoxide (CO), an oxidant and an amine, in the presence of a promoter.

According to a particular embodiment, the invention relates to the use, as defined above, of a Pd—X/CeO₂ catalyst, comprising palladium on a cerium dioxide support, in the implementation of a method for selectively preparing oxalates, from carbon monoxide (CO), an oxidant and an alcohol, at a pressure from 0.1 to 15 MPa.

According to a particular embodiment, the invention relates to the use, as defined above, of a Pd—X/CeO₂ catalyst, comprising palladium on a cerium dioxide support, in the implementation of a method for selectively preparing oxamides, from carbon monoxide (CO), an oxidant and an amine, at a pressure from 0.1 to 15 MPa.

Advantageously, the invention relates to the use as defined above in which molecular oxygen (O₂) or air is used as the oxidant.

According to a particular embodiment, the invention relates to the use as defined above of a Pd—X/CeO₂ catalyst, comprising palladium on a cerium dioxide support, in the implementation of a method for selectively preparing oxalates from carbon monoxide (CO), molecular oxygen (O₂) or air and an alcohol.

According to a particular embodiment, the invention relates to the use as defined above of a Pd—X/CeO₂ catalyst, comprising palladium on a cerium dioxide support, in the implementation of a method for selectively preparing oxamides from carbon monoxide (CO), molecular oxygen (O₂) or air and an amine.

According to a particular embodiment, the invention relates to the use as defined above of a Pd—X/CeO₂ catalyst, comprising palladium on a cerium dioxide support, in which the catalyst has a surface area, analysed by BET, from 50 to 250 m²/g, in particular from 100 to 250 m²/g, in particular from 100 to 200 m²/g, in the implementation of a method for selectively preparing oxalates from carbon monoxide, molecular oxygen or air and an alcohol.

According to a particular embodiment, the invention relates to the use as defined above of a Pd—X/CeO₂ catalyst, comprising palladium on a cerium dioxide support, in which the catalyst has a surface area, analysed by BET, from 50 to 250 m²/g, in particular from 100 to 250 m²/g, in particular from 100 to 200 m²/g, in the implementation of a method for selectively preparing oxamides from carbon monoxide, molecular oxygen or air and an amine.

According to a particular embodiment, the invention relates to the use as defined above of a Pd—X/CeO₂ catalyst, comprising palladium on a cerium dioxide support, in the implementation of a method for selectively preparing oxalates, from carbon monoxide (CO), molecular oxygen (O₂) or air and an alcohol, in the presence of a promoter.

According to a particular embodiment, the invention relates to the use as defined above of a Pd—X/CeO₂ catalyst, comprising palladium on a cerium dioxide support, in the implementation of a method for selectively preparing oxamides, from carbon monoxide (CO), molecular oxygen (O₂) or air and an amine, in the presence of a promoter.

According to a particular embodiment, the invention relates to the use as defined above of a Pd—X/CeO₂ catalyst, comprising palladium on a cerium dioxide support, in the implementation of a method for selectively preparing oxalates, from carbon monoxide (CO), molecular oxygen (O₂) or air and an alcohol, at a pressure from 0.1 to 15 MPa.

According to a particular embodiment, the invention relates to the use as defined above of a Pd—X/CeO₂ catalyst, comprising palladium on a cerium dioxide support, in the implementation of a method for selectively preparing oxamides, from carbon monoxide (CO), molecular oxygen (O₂) or air and an amine, at a pressure from 0.1 to 15 MPa.

The use of the invention is characterised in particular by the catalyst used.

### Specific Surface Area of the Catalyst Used

The specific surface area of the catalyst seems a priori to be one of the essential parameters for use according to the invention.

According to a particular embodiment, the invention relates to the use as defined above, in which the catalyst has an average surface area, analysed by BET, from 50 to 250 m²/g, in particular from 100 to 250 m²/g, especially from 100 to 200 m²/g.

The catalyst support used

According to a particular embodiment, the invention relates to the use as defined above, in which the said support used before impregnation of the palladium has a surface area from 100 to 300 m²/g, in particular from 150 to 160 m²/g.

The expression “from 100 to 300 m²/g” corresponds to the following ranges: from 100 to 125 m²/g; from 125 to 150 m²/g; from 150 to 175 m²/g; from 175 to 200 m²/g; from 200 to 225 m²/g; from 225 to 250 m²/g; from 250 to 275 m²/g; from 275 to 300 m²/g.

By “Palladium impregnation” it is meant the deposit of palladium atoms on the surface of the substrate. Impregnation can be achieved by bringing a palladium salt solution into contact with a substrate.

By “support used” it is meant the raw solid support used during the palladium impregnation step prior to the calcination step.

According to a particular embodiment, the invention relates to the use as defined above, in which said support used without impregnation of the palladium, therefore without the presence of palladium, after calcination at a temperature from 800 to 900° C. for a period of 2 h to 5 h has a surface area from 40 to 60 m²/g, in particular from 45 to 55 m²/g.

The expression “from 40 to 60 m²/g” corresponds to the following ranges: from 40 to 45 m²/g; from 45 to 50 m²/g; from 50 to 55 m²/g; from 55 to 60 m²/g.

By “calcination” it is meant an operation consisting of heating the solid support in ambient air in a closed enclosure to a high temperature of around 400 to 1000° C. in order to activate it or modify the physical characteristics of the support.

According to a particular embodiment, the invention relates to the use as defined above, in which said support used has a median pore size (D50) from 5 to 20 μm, in particular from 6 to 12 μm.

The median pore size can be analysed using known methods such as laser granulometry.

By “from 5 to 20 μm” it is meant the following ranges: from 5 to 10 μm; from 10 to 15 μm; from 15 to 20 μm.

According to a particular embodiment, the invention relates to the use as defined above, in which said support used has a loss on ignition (LOI) of less than 8%.

### Characteristics of Palladium Atoms

According to a particular embodiment, the invention relates to the use as defined above, in which the palladium/cerium dioxide catalyst comprises palladium atoms in a +2 oxidation state.

According to a particular embodiment, the invention relates to the use as defined above, in which the catalyst has a palladium content from 0.1 to 10%, in particular 2% or 5%, by weight relative to the total weight of the catalyst.

By “from 0.1 to 10%” it is meant the following ranges: from 0.1 to 0.5%; from 0.5 to 1%; from 1 to 2%; from 2 to 3%; from 3 to 4%; from 4 to 5%; from 5 to 6%; from 6 to 7%; from 7 to 8%; from 8 to 9%; from 9 to 10%.

### Presence of Doping Agents

By “dopant” it is meant a chemical element in the catalyst material other than palladium and cerium dioxide which improves the physical and chemical properties of the catalyst and improves the catalytic activity of the catalyst.

The doping element can either be in association with the palladium atoms and/or in association with the CeO₂.

Generally speaking, when the dopant is a transition metal, it improves the catalytic properties of the palladium. When the dopant is a species other than a transition metal, it modifies the surface properties of the support, which can improve the catalytic activity.

According to a particular embodiment, the dopant is selected from the following group of elements: Mn, Mg, Ca, Fe, Ba, Sr, Y, Nb, Zn, Bi, Sn, La, Pr, Nb and Sm.

In another particular embodiment, the dopant is Mn.

According to another particular embodiment, the dopant is a transition metal or a poor metal or a lanthanide, in particular chosen from Mn, Fe, Zn, Y, Nd, Zn, Bi, Sn, La, Pr, Nd and Sm. According to another particular embodiment, the dopant is an alkaline earth metal, in particular chosen from Mg, Ca, Ba and Sr.

According to a particular embodiment, the invention relates to the use as defined above, in which the catalyst has a dopant content from 0.5 and 10%, in particular 1%, by weight relative to the total weight of the catalyst.

By “from 0.5 to 10%” it is meant the following ranges: from 0.5 to 1%; from 1 to 2%; from 2 to 3%; from 3 to 4%; from 4 to 5%; from 5 to 6%; from 6 to 7%; from 7 to 8%; from 8 to 9%; from 9 to 10%.

According to another particular embodiment, the invention relates to the use according to the invention defined above, comprising a dopant chosen from Mn, Mg, Ca, Fe, Ba, Sr, Y, Nb, Zn, Bi, Sn, La, Pr, Nb and Sm, in particular at a content from 0.5 and 10%, preferably 1%, by weight relative to the total weight of the catalyst.

### Structure

According to a particular embodiment, the invention relates to the use as defined above, in which the catalyst has a fluorine-type structure by XRD and a crystallite size of less than 30 nanometres according to the Scherrer formula, in particular less than 20 nanometres, preferably less than 10 nanometres.

According to a particular embodiment, the invention relates to the use as defined above, in which the catalyst has a fluorine-type structure by XRD and a crystallite size of 1 to 30 nanometres according to the Scherrer formula, in particular 1 to 20 nanometres, preferably 1 to 10 nanometres.

The “less than 30 nanometres” range includes the following ranges: less than 25 nm; less than 20 nm; less than 15 nm; less than 12 nm; less than 10 nm; less than 9 nm; less than 8 nm; less than 7 nm; less than 6 nm; less than 5 nm; less than 4 nm; less than 3 nm; less than 2 nm; less than 1 nm.

The range “from 1 to 30 nanometres” includes the following ranges: from 1 to 2 nm; from 2 to 3 nm; from 3 to 4 nm; from 4 to 5 nm; from 5 to 6 nm; from 6 to 7 nm; from 7 to 8 nm; from 8 to 9 nm; from 9 to 10 nm; from 10 to 12 nm; from 12 to 15 nm; from 15 to 20 nm; from 20 to 25 nm; from 25 to 30 nm.

The XRD diffraction pattern of the catalyst shows a main contribution of the cerium dioxide support, the contribution from the palladium nanoparticles being negligible due to their concentration and size.

According to a particular embodiment, the invention relates to the use as defined above, in which the catalyst has a crystallinity level of 0 to 50%, preferably 0 to 20%.

### Morphology

According to a particular embodiment, the invention relates to the use as defined above, in which said catalyst is in the form of particles of average micrometric size, in particular from 1 to 100 μm.

By way of a non-limiting example, morphology and average size can be assessed by scanning electron microscopy (SEM).

### Nature of Pd on the Surface of the Catalyst

According to a particular embodiment, the invention relates to the use as defined above, in which the surface of said catalyst, analysed by XPS, comprises from 90 to 100% palladium in the oxidation state (II), in particular in the form of Pd—O or a PdₓCe₁₋ₓO₂ solid solution, x varying from 0.01 to 1.

According to a particular embodiment, the invention relates to the use as defined above, in which the catalyst has a fluorine-type structure by XRD and a crystallite size of less than 30 nanometres according to the Scherrer formula, in particular less than 20 nanometres, preferably less than 10 nanometres.
and/or in which the catalyst has a crystallinity of 0 to 50%, preferably 0 to 20%,
and/or in which said catalyst is in the form of particles of average micrometric size, in particular from 1 to 100 μm,
and/or in which the surface of the said catalyst, analysed by XPS, comprises from 90 to 100% palladium in the oxidation state (II), in particular in the form of Pd—O or a PdₓCe₁₋ₓO₂ solid solution, x varying from 0.01 to 1.

### Method

Another object of the present invention concerns the method for preparing an oxalate compound or an oxamide compound.

The invention concerns a method for preparing oxalates and oxamides combining the use of a Pd—X catalyst with a surface area, analysed by BET, of 50 to 250 m²/g, the presence of a promoter and the implementation at a pressure of 0.1 to 15 MPa.

The invention concerns a method for preparing oxalates and oxamides combining the use of a Pd—X catalyst with a surface area, analysed by BET, of 100 to 250 m²/g, the presence of a promoter and the implementation at a pressure of 0.1 to 15 MPa.

The invention concerns a method for preparing oxalates and oxamides combining the use of a Pd—X catalyst with a surface area, analysed by BET, of 100 to 200 m²/g, the presence of a promoter and the implementation at a pressure of 0.1 to 15 MPa.

The invention relates to the method for preparing an oxalate compound or an oxamide compound comprising a step A of bringing an alcohol or an amine, respectively, into contact with:
carbon monoxide,
an oxidising agent,
a catalyst of formula Pd—X/CeO₂, in which X represents the empty set or a dopant element, comprising Pd atoms on a CeO₂ support, in which the catalyst has a surface area, analysed by BET, of from 50 to 250 m²/g, in particular from 100 to 250 m²/g, preferably from 100 to 200 m²/g,
optionally a promoter,
optionally a base,
optionally a solvent,
to obtain a reaction medium which may be pressurised to 0.1 to 15 MPa,
optionally a step B of heating the reaction medium,
   to obtain the oxalate compound or the oxamide compound.

Advantageously, the invention relates to a method as defined above in which molecular oxygen (O₂) or air is used as the oxidant.

The invention relates to the method for preparing an oxalate compound or an oxamide compound comprising
a step A of contacting an alcohol or an amine, respectively, with:
   carbon monoxide,
   oxygen or air,
   a promoter, and
   a Pd—X/CeO₂ catalyst, comprising Pd atoms on a CeO₂ support, in which the catalyst has a surface area, analysed by BET, of from 50 to 250 m²/g, in particular from 100 to 250 m²/g, preferably from 100 to 200 m²/g,
   optionally a base,
   optionally a solvent,
to obtain a reaction medium,
optionally a step B of heating said reaction medium,
   to obtain the oxalate compound or the oxamide compound.

The invention relates to the method for preparing an oxalate compound or an oxamide compound comprising
a step A of contacting an alcohol or an amine, respectively, with:
   carbon monoxide,
   oxygen or air,
   a Pd—X/CeO₂ catalyst, comprising Pd atoms on a CeO₂ support, in which the catalyst has a surface area, analysed by BET, of from 50 to 250 m²/g, in particular from 100 to 250 m²/g, preferably from 100 to 200 m²/g,
   optionally a base,
   optionally a solvent,
to obtain a reaction medium,
optionally a step B of heating said reaction medium,
   to obtain the oxalate compound or the oxamide compound.

The invention relates to the method for preparing an oxalate compound or an oxamide compound comprising
a step A of contacting an alcohol or an amine, respectively, with:
   carbon monoxide,
   oxygen or air,
   a promoter
   a Pd—X/CeO₂ catalyst, comprising Pd atoms on a CeO₂ support,
   optionally a base,
   optionally a solvent,
to obtain a reaction medium,
and optionally a step B of heating said reaction medium,
   to obtain the oxalate compound or the oxamide compound.

By “reaction medium” it is meant all the species brought together during a chemical reaction. It includes in particular the reactants in liquid or gaseous form, the catalyst, and optionally a solvent, additives or promoters.

According to a particular embodiment, the invention relates to a method for preparing, as defined above, an oxalate compound or an oxamide compound, in which said reaction medium is pressurised to 0.1 to 15 MPa, in particular to a pressure from 0.1 to 10 MPa, preferably to 8.0 MPa.

According to a particular embodiment, the invention relates to a method for preparing an oxalate compound or an oxamide compound comprising
a step A of contacting an alcohol or an amine, respectively, with:
   carbon monoxide,
   oxygen or air,
   a promoter, and
   a Pd—X/CeO₂ catalyst,
   optionally a base,
   optionally a solvent,
to obtain a reaction medium pressurised to 0.1 to 15 MPa; in particular at a pressure of 0.1 to 10 MPa, preferably 8 MPa,
optionally a step B of heating the reaction medium,
   to obtain the oxalate compound or the oxamide compound.

According to a particular embodiment, the invention relates to a method for preparing an oxalate compound or an oxamide compound comprising
a step A of contacting an alcohol or an amine, respectively, with:
   carbon monoxide,
   oxygen or air,
   a Pd—X/CeO₂ catalyst, in which the catalyst has a surface area, analysed by BET, from 50 to 250 m²/g, in particular from 100 to 250 m²/g, preferably from 100 to 200 m²/g,
   optionally a base,
   optionally a solvent,
to obtain a reaction medium pressurised from 0.1 to 15 MPa; in particular from 0.1 to 10 MPa, preferably 8 MPa;
optionally a step B of heating said reaction medium,
   to obtain the oxalate compound or the oxamide compound.

According to a particular embodiment, the invention relates to a method for preparing an oxalate compound or an oxamide compound comprising
a step A of contacting an alcohol or an amine, respectively, with:
   carbon monoxide,
   oxygen or air,
   a promoter, and
   a Pd—X/CeO₂ catalyst, in which the catalyst has a surface area, analysed by BET, from 50 to 250 m²/g, in particular from 100 to 250 m²/g, preferably from 100 to 200 m²/g,
   optionally a base,
   optionally a solvent,
to obtain a reaction medium pressurised from 0.1 to 15 MPa; in particular from 0.1 to 10 MPa, preferably 8 MPa,
optionally a step B of heating said reaction medium,
   to obtain the oxalate compound or the oxamide compound.

The reaction medium is contained in a reactor.

The reaction medium is pressurised in a hermetically closed reactor, and in particular a sealed one.

In a particular embodiment, the reactor comprises the reaction mixture which is purged with nitrogen and/or dioxygen before the gaseous reactants (CO and O₂) are introduced.

By “reaction mixture” it is meant all the species in solid or liquid form in the method, excluding the gases. Thus the reaction mixture comprises the substrate (alcohol or amine), the catalyst, optionally a base, the promoter and optionally a solvent, but does not include the reagents in gaseous form such as carbon monoxide CO and oxygen O₂.

In a particular embodiment, the reaction medium is pressurised by the introduction of gaseous reactants comprising CO and optionally 02.

According to a particular embodiment, the invention relates to a method for preparing, as defined above, of an oxalate compound comprising a step A of bringing an alcohol into contact with:
carbon monoxide,
oxygen or air,
a promoter, and
a Pd—X/CeO₂ catalyst,
optionally a base
optionally a solvent,
to obtain a reaction medium,
optionally a step B of heating said reaction medium,
   to obtain the oxalate compound.

According to a particular embodiment, the invention relates to a method for preparing as defined above of an oxamide compound comprising:
a step A of contacting an amine with:
   carbon monoxide,
   oxygen or air,
   a promoter, and
   a Pd—X/CeO₂ catalyst,
   optionally a base
   optionally a solvent,
to obtain a reaction medium,
optionally a step B of heating said reaction medium,
   to obtain the oxamide compound.

According to a particular embodiment, the invention relates to a method as defined above for preparing an oxalate compound or an oxamide compound comprising a step A of bringing an alcohol or an amine, respectively, into contact with:
carbon monoxide, in particular used from 0.5 to 8.0 MPa, in particular at 6.5 MPa,
oxygen or air, in particular oxygen used at a rate from 0.5 to 2.5 MPa, in particular 1.5 MPa,
a promoter, in particular an iodine compound, chosen in particular from tetramethylammonium iodide, potassium iodide or sodium iodide, preferably tetramethylammonium iodide, preferably in a proportion from 0.1 to 5 mol % relative to the alcohol or amine, in particular in a proportion of 0.2 mol %,
a Pd—X/CeO₂ catalyst, comprising Pd atoms on a CeO₂ support, of formula Pd—X/CeO₂ in which X represents the empty set or a doping element, in which the catalyst has a surface area, analysed by BET, from 50 to 250 m²/g, in particular from 100 to 250 m²/g, preferably from 100 to 200 m²/g, preferably the palladium is in an amount from 0.01 to 10 mol % relative to the alcohol or the amine,
optionally a base, in particular triethylamine, preferably used in a proportion from 0.1 to 5 mol % relative to the alcohol or amine, in particular in a proportion of 0.15 mol %,
optionally a solvent, in particular selected from acetonitrile, tetrahydrofuran, dioxane and toluene, preferably acetonitrile,
to obtain a reaction medium,
optionally a step B of heating said reaction medium,
   to obtain the oxalate compound or the oxamide compound.

### Method for Preparing Oxalates

### Heating Stage

Advantageously, the method for preparing oxalate involves a heating step.

According to a particular embodiment, the invention relates to a method for preparing, as defined above, an oxalate compound comprising
a step A of bringing an alcohol or an amine into contact with:
   carbon monoxide,
   oxygen or air,
   a promoter, and
   a Pd—X/CeO₂ catalyst
   optionally a base,
   optionally a solvent,
to obtain a reaction medium,
a step B of heating said reaction medium,
   to obtain the oxalate compound.

According to a particular embodiment, the invention relates to a method for preparing, as defined above, an oxalate compound, in which the heating step B is carried out at a temperature from 25 to 200° C., in particular from 60 to 110° C., preferably about 90° C.

The expression “from 25 to 200° C.” corresponds to the following ranges: from 25 to 40° C.; from 40 to 60° C.; from 60 to 80° C.; from 80 to 100° C.; from 100 to 120° C.; from 120 to 140° C.; from 140 to 160° C.; from 160 to 180° C.; from 180 to 200° C.

The expression “from 60 to 110° C.” corresponds to the following ranges: from 60 to 70° C.; from 70 to 80° C.; from 80 to 90° C.; from 90 to 100° C.; from 100 to 110° C.

Advantageously, the method for preparing an oxalate compound as defined above is carried out at a temperature of about 90° C.

According to a particular embodiment, the invention relates to a method for preparing an oxalate compound comprising a step A of bringing an alcohol into contact with:
carbon monoxide,
oxygen or air,
a promoter,
a Pd—X/CeO₂ catalyst, comprising Pd atoms on a CeO₂ support, of formula Pd—X/CeO₂ in which X represents the empty set or a doping element, in which the catalyst has a surface area, analysed by BET, from 50 to 250 m²/g, in particular from 100 to 250 m²/g, preferably from 100 to 200 m²/g, preferably the palladium is in an amount of from 0.01 to 10 mol % relative to the alcohol or the amine,
optionally a base,
optionally a solvent,
to obtain a reaction medium,
a step B of heating said reaction medium, in particular at a temperature from 25 to 200° C., in particular from 60 to 110° C., preferably about 90° C., to obtain the oxalate compound.

### Solvent

The method according to the invention for preparing oxalates can be carried out with or without a solvent in the reaction medium.

According to a particular embodiment, the invention relates to a method for preparing as defined above, in the presence of a solvent.

The presence of solvent in the reaction medium improves the reactivity of the method according to the invention.

According to a particular embodiment, the invention relates to a method for preparing, as defined above, of an oxalate compound comprising a step A of bringing an alcohol into contact with:
carbon monoxide,
oxygen or air,
a promoter,
a Pd—X/CeO₂ catalyst, and
a solvent,
optionally a base
to obtain a reaction medium,
optionally a step B of heating the reaction medium,
   to obtain the oxalate compound.

According to a particular embodiment, the invention relates to method for preparing, as defined above, an oxalate compound comprising a step A of bringing an alcohol into contact with:
carbon monoxide,
oxygen or air,
a base,
a promoter,
a Pd—X/CeO₂ catalyst, and
a solvent,
to obtain a reaction medium,
optionally a step B of heating the reaction medium,
   to obtain the oxalate compound.

According to a particular embodiment, the invention relates to a method for preparing, as defined above, an oxalate compound comprising a step A of bringing an alcohol into contact with:
carbon monoxide,
oxygen or air,
a promoter,
a Pd—X/CeO₂ catalyst, and
a solvent,
to obtain a reaction medium,
optionally a step B of heating the reaction medium,
   to obtain the oxalate compound.

### Solvent-Free

Advantageously, the method for preparing oxalate can be carried out without solvent.

According to a particular embodiment, the invention relates to the method for preparing as defined above, carried out in the absence of a solvent. Alcohol can act as both solvent and reagent.

This limits the preparation steps and the presence of degradation products to be treated, and limits the solvent treatment steps. The alcohol can be easily recycled if required, and a solvent/alcohol separation step is not necessary.

According to a particular embodiment, the invention relates to a method for preparing, as defined above, an oxalate compound comprising a step A of bringing an alcohol into contact with:
carbon monoxide,
oxygen or air,
a promoter, and
a Pd—X/CeO₂ catalyst and
optionally a base
to obtain a reaction medium,
and optionally a step B of heating said reaction medium,
   to obtain the oxalate compound.

According to a particular embodiment, the invention relates to a method for preparing, as defined above, an oxalate compound comprising a step A of bringing an alcohol into contact with:
carbon monoxide,
oxygen or air,
a base,
a promoter, and
a Pd—X/CeO₂ catalyst
to obtain a reaction medium,
and optionally a step B of heating said reaction medium,
   to obtain the oxalate compound.

According to a particular embodiment, the invention relates to a method for preparing, as defined above, an oxalate compound comprising a step A of bringing an alcohol into contact, respectively, with:
carbon monoxide,
oxygen or air,
a promoter, and
a Pd—X/CeO₂ catalyst,
to obtain a reaction medium,
and optionally a step B of heating said reaction medium,
   to obtain the oxalate compound.

### Alcohol

Provided that it is possible to react with CO by carbonylation, the choice of R—OH alcohol used to prepare oxalates is not limited. Preferably one and the same alcohol is chosen in a reaction, in order to prepare oxalates with the same R group.

According to a particular embodiment, the invention relates to a method for preparing, as defined above, an oxalate compound of Formula 2, in which step A comprises contacting an alcohol of Formula 1:

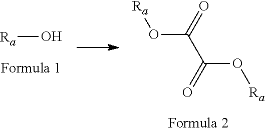

in which Rₐ represents:
a C₁ to C₂₀ linear or branched alkyl group,
a C₃ to C₁₀ cycloalkyl group,
a C₅ to C₂₀ linear or branched alkyl-aryl or alkyl-heteroaryl group.

For the purposes of this invention, by “C₁ to C₂₀ linear or branched alkyl group” it is meant a saturated, linear or branched, acyclic carbon chain comprising 1 to 20 carbon atoms. These groups are methyl, ethyl, propyl, butyl, pentyl, hexyl, heptyl, octyl, nonyl, decyl, undecyl, dodecyl, tridecyl, tetradecyl, pentadecyl, cetyl, heptadecyl, octadecyl, nonadecyl and eicosyl.

The definition of alkyl includes all possible isomers. For example, the term butyl includes n-butyl, iso-butyl, sec-butyl and ter-butyl. One or more hydrogen atoms may be replaced in the alkyl chain.

By “C₃ to C₁₀ cycloalkyl” it is meant: a C₃ cyclopropyl group, a C₄ cyclobutyl group, a C₅ cyclopentyl group, a C₆ cyclohexyl group, a C₇ cycloheptyl group, a C₈ cyclooctyl group, a C₉ cyclononyl group, or a C₁₀ cyclodecyl group, and fused cycloalkane rings such as adamantyl.

By “C₅ to C₂₀ alkylaryl” it is meant a group consisting of a linear or branched alkyl chain linked to an aromatic group, the alkylaryl group comprising 5 to 20 carbon atoms. The aryl groups according to the present invention can also be substituted, in particular by one or more substituents chosen from a C₁ to C₁₀ linear or branched alkyl group.

Phenyl, toluyl, anisyl and naphthyl o-tolyl, m-tolyl, p-tolyl, o-xylyl, m-xylyl, p-xylyl, are examples of aryl groups.

The term “heteroaryl” refers to an aryl group as defined above, comprising atoms other than carbon atoms, in particular N, O or S within the aromatic ring. Pyridyl, imidazoyl, furfuryl or furanyl are examples of heteroaryl groups according to the present invention.

According to a particular embodiment, the invention relates to a method for preparing, as defined above, an oxalate compound, in which step A comprises bringing an alcohol chosen from methanol, ethanol and isopropanol into contact.

According to a particular embodiment, the invention relates to a method for preparing, as defined above, an oxalate compound of Formula 2, in which step A comprises contacting an alcohol of Formula 1:

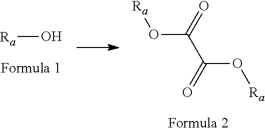

in which Rₐ represents:
a C₁ to C₂₀ linear or branched alkyl group,
a C₃ to C₁₀ cycloalkyl group,
a C₅ to C₂₀ alkyl-aryl or alkyl-heteroaryl group, in particular the alcohol is chosen from methanol, ethanol and isopropanol.

### Method for Preparing Oxamides

The method according to the invention for preparing oxamides can be carried out with or without a step for heating the reaction medium, preferably without heating the reaction medium.

### Heating Stage

According to a particular embodiment, the invention relates to a method for preparing an oxamide compound as defined above, comprising a step of heating the reaction medium. According to a particular embodiment, the invention relates to a method for preparing, as defined above, an oxamide compound comprising a step A of bringing an amine into contact with:
carbon monoxide,
oxygen or air,
a promoter,
a Pd—X/CeO₂ catalyst,
a solvent, and
optionally a base,
to obtain a reaction medium,
   and a step B of heating said reaction medium,
   to obtain the oxamide compound.

According to a particular embodiment, the invention relates to a method for preparing, as defined above, an oxamide compound comprising a step A of bringing an amine into contact with:
carbon monoxide,
oxygen or air,
a base,
a promoter,
a Pd—X/CeO₂ catalyst, and
a solvent,
to obtain a reaction medium,
   and a step B of heating said reaction medium,
   to obtain the oxamide compound.

According to a particular embodiment, the invention relates to a method for preparing, as defined above, an oxamide compound comprising a step A of bringing an amine into contact with:
carbon monoxide,
oxygen or air,
a promoter,
a Pd—X/CeO₂ catalyst, and
a solvent,
to obtain a reaction medium,
   and a step B of heating said reaction medium,
   to obtain the oxamide compound.

According to one embodiment, the method as defined above for preparing an oxamide compound comprises a step B of heating at a temperature from 25 to 200° C.

### Method Used at Ambient Temperature

Advantageously, the method for preparing an oxamide compound can be carried out at room temperature.

Ambient temperature” means a temperature from 20 to 25° C.

Consequently, heating the reaction medium is optional, which represents an industrial advantage in terms of cost and safety.

According to a particular embodiment, the invention relates to a method for preparing, as defined above, an oxamide compound prepared without heating the reaction medium.

According to a particular embodiment, the invention relates to a method as defined above for preparing an oxamide compound, in which said reaction medium is maintained at an ambient temperature of 20 to 25° C. during the reaction.

According to a particular embodiment, the invention relates to a method as defined above for preparing an oxamide compound comprising a step A of bringing an amine into contact with:
carbon monoxide,
oxygen or air,
a promoter,
a Pd—X/CeO₂ catalyst,
a solvent, and
optionally a base,
   to obtain a reaction medium comprising the oxamide compound.

According to a particular embodiment, the invention relates to a method as defined above for preparing an oxamide compound comprising a step A of bringing an amine into contact with:
carbon monoxide,
oxygen or air,
a base,
a promoter,
a Pd—X/CeO₂ catalyst, and
a solvent,
   to obtain a reaction medium comprising the oxamide compound.

According to a particular embodiment, the invention relates to a method as defined above for preparing an oxamide compound comprising a step A of bringing an amine into contact with:
carbon monoxide,
oxygen or air,
a promoter,
a Pd—X/CeO₂ catalyst, and
a solvent,
   to obtain a reaction medium comprising the oxamide compound.

According to a particular embodiment, the invention relates to a method for preparing, as defined above, an oxamide compound comprising a step A of bringing an amine into contact with:
carbon monoxide,
oxygen or air,
a promoter,
a Pd—X/CeO₂ catalyst, comprising Pd atoms on a CeO₂ support, of the formula Pd—X/CeO₂ in which X represents the empty set or a dopant element, in which the catalyst has a surface area from 50 to 250 m²/g, preferably from 0.01 to 10 mole % relative to the alcohol or amine,
a solvent,
optionally a base,
   to obtain a reaction medium comprising the oxamide compound.
   Method with/without a Base

The method according to the invention for preparing oxamides can be carried out with or without a base added to the reaction medium.

According to a particular embodiment, the invention concerns a method for preparing as defined above, in the presence of a base.

According to a particular embodiment, the invention relates to a method for preparing, as defined above, an oxamide compound comprising a step A of bringing an amine into contact with:
carbon monoxide,
oxygen or air,
a base,
a promoter,
a Pd—X/CeO₂ catalyst, and
a solvent,
to obtain a reaction medium,
optionally a step B of heating the reaction medium,
   to obtain the oxamide compound.

According to a particular embodiment, the invention relates to a preparation method as defined above, without any base added to the reaction medium, the amine having the role of reagent and base.

The absence of base in the reaction medium limits the number of reagents to be introduced into the method, limits the formation of degradation products and limits the separation and purification steps.

According to a particular embodiment, the invention relates to a method for preparing an oxamide compound as defined above, comprising a step A of bringing an amine into contact with:
carbon monoxide,
oxygen or air,
a promoter, and
a Pd—X/CeO₂ catalyst, and
a solvent,
to obtain a reaction medium,
optionally a step B of heating the reaction medium,
   to obtain the oxamide compound.

### Amines

Provided that it can react with CO, the choice of amine used to prepare oxamides is not limited. Preferably one and the same amine is chosen, in order to prepare symmetrical oxamides. According to a particular embodiment, the invention relates to a method for preparing, as defined above, an oxamide compound of Formula 4, in which step A comprises contacting an amine of Formula 3:

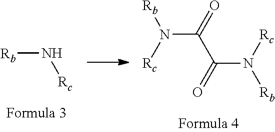

in which R_{b} and R_{c} independently of one another represent:
a hydrogen atom,
a C₁ to C₂₀ linear or branched alkyl group,
a C₅ to C₂₀ alkyl-aryl or alkyl-heteroaryl group,
a C₃ to C₁₀ cycloalkyl group,
   at least one of the groups R_{b} or R_{c} being different hydrogen,
   R_{b} and R_{c} can form a cycle.

In a particular embodiment, the groups R_{b} and R_{c} form a ring.

In a particular embodiment, the R groups_{b} and R_{c} are not linked and do not form a ring.

In a particular embodiment, the groups R_{b} and R_{c} are different.

In a particular embodiment, the groups R_{b} and R_{c} are identical.

According to a particular embodiment, the invention relates to a method for preparing an oxamide compound as defined above, in which step A comprises contacting an amine chosen from piperidine, pyrrolidine, butylamine, benzylamine, furfurylamine and cyclohexylamine.

According to a particular embodiment, the invention relates to a method for preparing, as defined above, an oxamide compound of Formula 4, in which step A comprises contacting an amine of Formula 3:

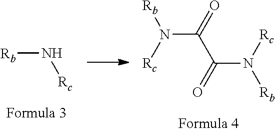

in which R_{b} and R_{c} independently of one another represent:
a hydrogen atom,
a C₁ to C₂₀ linear or branched alkyl group,
a C₃ to C₁₀ cycloalkyl group,
a C₅ to C₂₀ alkyl-aryl or alkyl-heteroaryl group,
   at least one of the groups R_{b} or R_{c} being different from hydrogen,
   R_{b} and R_{c} can form a cycle,
   in particular the amine is chosen from piperidine, pyrrolidine, butylamine, benzylamine, furfurylamine and cyclohexylamine.

### Common Features of the Method for Preparing Oxalates and Oxamides

### Catalyst Structure

According to a particular embodiment, the invention relates to a method for preparing, as defined above, an oxalate compound or an oxamide compound, in which the catalyst has a fluorine-type structure by XRD and a crystallite size of less than 30 nanometres according to the Scherrer formula, in particular less than 20 nanometres, preferably less than 10 nanometres. According to a particular embodiment, the invention relates to a method for preparing, as defined above, an oxalate compound or an oxamide compound, in which the catalyst has a fluorine-type structure by XRD and a crystallite size from 1 to 30 nanometres according to the Scherrer formula, in particular from 1 to 20 nanometres, preferably from 1 to 10 nanometres.

According to a particular embodiment, the invention relates to a method for preparing, as defined above, an oxalate compound or an oxamide compound, in which the catalyst has a degree of crystallinity of from 0 to 50%, preferably from 0 to 20%.

### Catalyst Morphology

According to a particular embodiment, the invention relates to a method for preparing, as defined above, an oxalate compound or an oxamide compound, in which the catalyst is in the form of particles of average micrometric size, in particular from 1 to 100 μm.

### Nature of Pd on the Surface of the Catalyst

According to a particular embodiment, the invention relates to a method for preparing, as defined above, an oxalate compound or an oxamide compound, in which the surface of the said catalyst, analysed by XPS, comprises from 90 to 100% palladium in the oxidation state (II), in particular in the form of Pd—O or a PdₓCe₁₋ₓO₂ solid solution.

According to a particular embodiment, the invention relates to a method for preparing, as defined above, of an oxalate compound or an oxamide compound,
in which the catalyst has a fluorine-type structure by XRD and a crystallite size of less than 30 nanometres according to the Scherrer formula, in particular less than 20 nanometres, preferably less than 10 nanometres
and/or in which the catalyst has a crystallinity from 0 to 50%, preferably from 0 to 20%, and/or in which the catalyst is in the form of particles of average micrometric size, in particular from 1 to 100 μm,
and/or in which the surface of the catalyst, analysed by XPS, comprises from 90 to 100% palladium in the oxidation state (II), in particular in the form of Pd—O or a PdₓCe₁₋ₓO₂ solid solution, x varying from 0.01 to 1.

According to a particular embodiment, the invention relates to a method for preparing, as defined above, an oxalate compound or an oxamide compound, in which the support used has a surface area from 100 to 300 m²/g, in particular from 150 to 160 m/g.²

According to a particular embodiment, the invention relates to a method for preparing, as defined above, an oxalate compound or an oxamide compound, in which the catalyst has a palladium content from 0.1 to 10%, in particular 2% or 5%, by weight relative to the total weight of the catalyst.

According to a particular embodiment, the invention relates to a method for preparing, as defined above, an oxalate compound or an oxamide compound, in which the catalyst additionally comprises a dopant.

According to a particular embodiment, the dopant is selected from the following group of elements: Mn, Mg, Ca, Fe, Ba, Sr, Y, Nb, Zn, Bi, Sn, La, Pr, Nb and Sm.

In a particular embodiment, the dopant is Mn.

According to a particular embodiment, said catalyst comprises a dopant content varying from 0.5 to 10%, in particular 1%, by weight relative to the total weight of the catalyst.

According to a particular embodiment, the invention relates to a method for preparing, as defined above, an oxalate compound or an oxamide compound, in which the base is triethylamine.

According to a particular embodiment, the invention relates to a method for preparing, as defined above, an oxalate compound or an oxamide compound, in which the promoter is an iodine compound, in particular a salt.

According to a particular embodiment, the invention relates to a method for preparing, as defined above, an oxalate compound or an oxamide compound, in which the promoter is tetrabutylammonium iodide, potassium iodide or sodium iodide, preferably tetrabutylammonium iodide.

According to a particular embodiment, the invention relates to a method for preparing, as defined above, an oxalate compound or an oxamide compound, said method being carried out in the absence of a solvent.

According to a particular embodiment, the invention relates to a method for preparing, as defined above, an oxalate compound or an oxamide compound, in which the solvent is acetonitrile, tetrahydrofuran, dioxane or toluene, preferably acetonitrile.

According to a particular embodiment, the invention relates to a method for preparing, as defined above, an oxalate compound or an oxamide compound, in which oxygen is used at a rate from 0.5 to 2.5 MPa (5 to 25 bars), in particular at 1.5 MPa (15 bars).

According to a particular embodiment, the invention relates to a method for preparing, as defined above, an oxalate compound or an oxamide compound, in which carbon monoxide is used at a rate from 0.5 to 8.0 MPa (50 to 80 bars), in particular at 6.5 MPa (65 bars).

According to a particular embodiment, the invention relates to a method for preparing, as defined above, an oxalate compound or an oxamide compound, in which:
oxygen is used at 0.5 to 2.5 MPa (5 to 25 bars), in particular at 1.5 MPa (15 bars),
and carbon monoxide is used from 0.5 to 8.0 MPa (50 to 80 bars), particularly at 6.5 MPa (65 bars).

According to a particular embodiment, the invention relates to a method for preparing as defined above, in which the reaction medium is pressurized solely by CO and O₂. It is understood that the pressure in the reactor is that arising from the gaseous reactants CO and O₂.

According to a particular embodiment, the invention relates to a method for preparing, as defined above, an oxalate compound or an oxamide compound, in which the carbon monoxide/oxygen pressure ratio used is from 3 to 10, in particular about 4.

The expression “from 3 to 10” corresponds to the ranges: from 3 to 4; from 4 to 5; from 5 to 6; from 6 to 7; from 7 to 8; from 8 to 9; from 9 to 10.

According to a particular embodiment, the invention relates to a method for preparing, as defined above, an oxalate compound or an oxamide compound, in which the base is used in a proportion from 0.1 to 5 mol % relative to the alcohol or amine, in particular in a proportion of 0.15 mol %.

The expression “from 0.1 to 5%” corresponds to the following ranges: from 0.1 to 0.15%; from 0.15 to 0.2; from 0.2 to 0.3%; from 0.3 to 0.4%; from 0.4 to 0.5; from 0.5 to 1%; from 1 to 2%; from 2 to 3%; from 3 to 4%; from 4 to 5%.

According to a particular embodiment, the invention relates to a method for preparing, as defined above, an oxalate compound or an oxamide compound, in which the promoter is used in a proportion of 0.1 to 5 mol % relative to the alcohol or amine, in particular in a proportion of 0.2 mol %.

The expression “from 0.1 to 5%” corresponds to the following ranges: from 0.1 to 0.15%; from 0.15 to 0.2%; from 0.2 to 0.3%; from 0.3 to 0.4%; from 0.4 to 0.5%; from 0.5 to 1%; from 1 to 2%; from 2 to 3%; from 3 to 4%; from 4 to 5%.

According to a particular embodiment, the invention relates to a method for preparing, as defined above, an oxalate compound or an oxamide compound, in which the catalyst is used in a proportion of 0.01 to 10 mol % relative to the alcohol or amine, in particular in a proportion of 0.15 mol %.

The expression “from 0.01 to 10%” corresponds to the following ranges: from 0.01 to 0.05%; from 0.05 to 0.1%; from 0.1 to 0.15%; from 0.15 to 0.2%; from 0.2 to 0.5%; from 0.5 to 1%; from 1 to 2%; from 2 to 3%; from 3 to 4%; from 4 to 5%; from 5 to 6%; from 6 to 7%; from 7 to 8%; from 8 to 9%; from 9 to 10%.

According to a particular embodiment, the invention relates to a preparation method as defined above, in which the reaction medium is heated to a temperature from 25 to 200° C., in particular from 60 to 110° C., in particular about 90° C., in the case of the preparation of oxalates, and to room temperature in the case of the preparation of oxamides, in particular for a period from 2 to 72 hours, in particular for 16 hours.

According to a particular embodiment, the invention relates to a method for preparing, as defined above, an oxalate compound or an oxamide compound, in which the method further comprises, after step A of contacting, a successive step C of filtering the reaction medium to obtain a recovered catalyst and a filtrate free of catalyst.

Advantageously, the catalyst recovered after the method of the invention is not degraded and is stable, and it can be reused in another catalytic reaction method. It is therefore possible to repeat the method according to the invention with the same recovered catalyst.

According to a particular embodiment, the invention relates to a method for preparing, as defined above, an oxalate compound or an oxamide compound, in which the catalyst is stable at the end of the reaction and can be reused in another catalytic reaction method.

According to a particular embodiment, the invention relates to a method for preparing, as defined above, an oxalate compound or an oxamide compound, in which the catalyst used in the contacting step A is a catalyst recovered at the end of the successive filtration step C. According to a particular embodiment, the invention relates to a preparation method as defined above, in which the method is carried out in continuous flow, the catalyst being a heterogeneous Pd—X/CeO₂ catalyst according to the invention.

According to a particular embodiment, the invention relates to a continuous flow process for preparing oxalates or oxamides as defined above,
in which the catalyst is a heterogeneous Pd—X/CeO₂ catalyst according to the invention introduced into a column or a cartridge,
or in which the catalyst is suspended in the reaction mixture.

By way of a non-limiting example, the continuous flow process is carried out in a reactor of the following type:
a continuously stirred tank reactor (CSTR),
a flow reactor or a tubular reactor,
a reactor with a fixed or packed bed.

By way of example, the method according to the invention can be implemented in a flow chemistry apparatus, for example in commercial reactors such as “H-Cube Pro®” or “Phoenix®” from ThalesNano INC. (7 Zahony Street, Graphisoft Park, Building D, H-1031 Budapest, Hungary) or such as the “E-Series” or “R-Series flow chemistry systems” from Vapourtec Ltd (Unit 21/Park Farm Business Centre/Fornham Pk, Bury Saint Edmunds IP28 6TS. United Kingdom).

Advantageously, the continuous flow process is carried out at a temperature from 25° C. to 200° C.

Advantageously, the continuous flow process is carried out at a pressure from 0.1 MPa to 15 MPa, in particular 0.1 to 4 MPa.

The expression “from 0.1 to 4 MPa” corresponds to the following ranges: from 0.1 to 0.5 MPa; from 0.5 to 1.0 MPa; from 1.0 to 1.5 MPa; from 1.5 to 2.0 MPa; from 2.0 to 2.5 MPa; from 2.5 to 3.0 MPa; from 3.0 to 3.5 MPa; from 3.5 to 4.0 MPa.

In a particular embodiment, the continuous flow method is carried out in a reactor in which the gases represent from 10 to 90% of the reactor volume.

The expression “from 10 to 90%” corresponds to the following ranges: from 10 to 20%; from 20 to 30%; from 30 to 40%; from 40 to 50%; from 50 to 60%; from 60 to 70%; from 70 to 80%; from 80 to 90%.

According to a particular embodiment, the continuous flow process is carried out by means allowing a contact time between the reagents from 1 second to 2 hours, in particular from 1 second to 2 minutes.

The expression “from 1 second to 2 hours” corresponds to the following ranges: from 1 to 15 seconds; from 15 to 30 seconds; from 30 seconds to 1 minute; from 1 to 2 minutes; from 2 to 15 minutes; from 15 to 30 minutes; from 30 minutes to 1 hour; from 1 to 2 hours.

In a particular embodiment, the continuous flow process comprises means for introducing into the reactor the stream of CO in contact with the substrate (the alcohol or the amine) and the stream of oxygen or air individually or as a mixture.

According to a particular embodiment, the invention relates to a method for preparing, as defined above, an oxalate compound or an oxamide compound, additionally comprising a purification step, in which the oxalate product or the oxamide product is isolated from carbonate products or urea products respectively, in an oxalate/carbonate ratio greater than 98%, or in an oxamide/urea ratio greater than 98%.

According to a particular embodiment, the invention concerns a method for preparing as defined above, in which the oxalate product is isolated from carbonate products, the oxalate/carbonate ratio being greater than 98%.

According to a particular embodiment, the invention concerns a method for preparing as defined above, in which the oxamide product is isolated from urea products, the oxamide/urea ratio being greater than 98%.

By way of non-limiting examples, the oxalate product or the oxamide product can be isolated by distillation or by extraction and recrystallisation in the purification stage.

According to a particular embodiment, the invention relates to a method for preparing, as defined above, an oxalate compound or an oxamide compound, in which the selectivity towards the oxalate product, or the oxamide product, is greater than 80%, in particular greater than 85%. According to a particular embodiment, the invention relates to a method for preparing, as defined above, of an oxalate compound in which the selectivity towards the oxalate product is greater than 80%, in particular about 85%.

According to a particular embodiment, the invention relates to a method for preparing an oxamide compound as defined above, in which the selectivity towards the oxamide product is greater than 95%, in particular about 99%.

The method according to the invention for an oxalate compound, for example, has a selectivity of around 85%.

The method according to the invention of an oxamide compound has a selectivity of around 99%.

The yields of the method for preparing oxalates or oxamides according to the invention can be described in terms of “Number of Catalytic Cycles (NCC)”.

In particular, the catalytic activity under given conditions of temperature, pressure and concentrations of solutes and time of the method for preparing oxalates or oxamides according to the invention can be described in terms of NCC.

The “Number of Catalytic Cycles (NCC)” is defined as the ratio between the number of moles of product formed (nprod) and the number of moles of palladium in the catalyst (ncat):

$$NCC = \frac{{n}_{prod}}{{n}_{cat}}$$

Unlike the Turnover Number (TON), which represents the maximum number of catalytic cycles that a catalyst can achieve before its total and irreversible degradation, the Catalytic Cycle Number represents the total number of catalytic cycles achieved by the catalyst under given reaction conditions. At the end of the reaction, the catalyst used would not necessarily be degraded and could therefore be reused. The NCC is therefore not a measure of the lifetime of a catalyst, but a measure of the productivity of the catalyst under the given conditions of the catalysed reaction.

### Catalyst

Another object of the present invention is a Pd—X/CeO₂ catalyst, comprising palladium on a cerium dioxide support,
in which the catalyst has a surface area, analysed by BET, from 50 to 250 m²/g, in particular from 100 to 250 m²/g, preferably from 100 to 200 m²/g.
and in which the catalyst has a palladium content from 0.1 to 10%, in particular 2% or 5%, by weight relative to the total weight of the catalyst.

In a particular embodiment, the invention concerns a Pd—X/CeO₂ catalyst as defined above, comprising palladium on a cerium dioxide support,

in which the catalyst has a surface area, analysed by BET, from 100 to 250 m²/g, in particular from 100 to 200 m²/g,
and in which the catalyst has a palladium content from 0.1 to 10%, in particular 2% or 5%, by weight relative to the total weight of the catalyst.

According to a particular embodiment, the invention relates to a Pd—X/CeO₂ catalyst as defined above, in which the catalyst has a fluorine-type structure by XRD and a crystallite size of less than 30 nanometres according to the Scherrer formula, in particular less than 20 nanometres, preferably less than 10 nanometres.

According to a particular embodiment, the invention relates to a Pd—X/CeO₂ catalyst as defined above, in which the catalyst has a fluorine-type structure by XRD and a crystallite size from 1 to 30 nanometres according to the Scherrer formula, in particular from 1 to 20 nanometres, preferably from 1 to 10 nanometres.

In a particular embodiment, the invention concerns a Pd—X/CeO₂ catalyst as defined above, comprising palladium on a cerium dioxide support,
in which the catalyst has:
   a surface area, analysed by BET, from 100 to 250 m²/g, in particular from 100 to 200 m²/g,
   and/or a fluorite-type structure by XRD and a crystallite size of less than 30 nanometres according to the Scherrer formula, in particular less than 20 nanometres, preferably less than 10 nanometres,
and in which the catalyst has a palladium content from 0.1 to 10%, in particular 2% or 5%, by weight relative to the total weight of the catalyst.

The crystalline structure of the catalyst can be analysed by X-ray powder diffraction. The structure is analysed by assigning the diffraction peaks present in the diffraction pattern obtained by comparison with JCPDS type files. The fluorite structure of cerium oxide is shown in JCPDS file 34-0394.

The size of the crystallites was evaluated using the following Scherrer formula:

$$\begin{matrix}t = \frac{k . λ}{H . cos θ} & t = crystallite size \\ & k = correction factor = 0.89 \\ & λ = wavelength of the source \\ & H = length at the halfway point of the peak \\ & θ = diffraction angle\end{matrix}$$

The “less than 30 nanometres” range includes the following ranges: less than 25 nm; less than 20 nm; less than 15 nm; less than 12 nm; less than 10 nm; less than 9 nm; less than 8 nm; less than 7 nm; less than 6 nm; less than 5 nm; less than 4 nm; less than 3 nm; less than 2 nm; less than 1 nm.

The “less than 10 nanometres” range includes 1 to 5 nm and 5 to 10 nm.

The range “from 1 to 30 nanometres” includes the following ranges: from 1 to 2 nm; from 2 to 3 nm; from 3 to 4 nm; from 4 to 5 nm; from 5 to 6 nm; from 6 to 7 nm; from 7 to 8 nm; from 8 to 9 nm; from 9 to 10 nm; from 10 to 12 nm; from 12 to 15 nm; from 15 to 20 nm; from 20 to 25 nm; from 25 to 30 nm.

The catalysts of the invention have a fluorine-type structure of cerium oxide identical to that of the cerium oxide support used in its preparation.

The catalysts of the invention have the advantage of low crystallinity, indicated by a small crystallite size, in particular 1 to 10 nanometres.

According to a particular embodiment, the invention relates to a Pd—X/CeO₂ catalyst as defined above, in which the catalyst has a crystallinity level of 0 to 50%, preferably 0 to 20%.

In a particular embodiment, the invention concerns a Pd—X/CeO₂ catalyst as defined above, comprising palladium on a cerium dioxide support,
in which the catalyst has:
   a surface area, analysed by BET, from 100 to 250 m²/g, in particular from 100 to 200 m²/g,
   and/or a crystallinity level from 0 to 50%, preferably from 0 to 20%.

The range “from 0 to 50%” includes the following ranges: from 0 to 10%; from 10 to 20%; from 20 to 30%; from 30 to 40%; from 40 to 50%.

According to a particular embodiment, the invention relates to a Pd—X/CeO₂ catalyst as defined above, in which said catalyst is in the form of particles of average micrometric size, in particular from 1 to 100 μm.

In a particular embodiment, the invention concerns a Pd—X/CeO₂ catalyst as defined above, comprising palladium on a cerium dioxide support, in which the catalyst has a surface area, analysed by BET, from 100 to 250 m²/g, in particular from 100 to 200 m²/g,
in which the catalyst has a palladium content from 0.1 to 10%, in particular 2% or 5%, by weight relative to the total weight of the catalyst,
   and the said catalyst is in the form of particles of average micrometric size, in particular from 1 to 100 μm.

Advantageously, the catalysts according to the invention are in the form of a population of particles of average micrometric size, making them easier to handle and avoiding the safety conditions that apply to nanometric particles.

By “average micrometric size” it is meant an average size from 1 to 1000 μm.

The 1 to 100 μm range includes the following ranges: from 1 to 10 μm; from 10 to 50 μm; from 50 to 75 μm; from 75 to 100 μm.

According to a particular embodiment, the invention relates to a Pd—X/CeO₂ catalyst as defined above, in which the surface of the said catalyst, analysed by XPS, comprises from 90 to 100% palladium in the oxidation state (II), in particular in the form of Pd—O or a PdₓCe₁₋ₓO₂ solid solution, x varying from 0.01 to 1.

In a particular embodiment, the invention concerns a Pd—X/CeO₂ catalyst as defined above, comprising palladium on a cerium dioxide support,

in which the catalyst has a surface area, analysed by BET, from 100 to 250 m²/g, in particular from 100 to 200 m²/g,
in which the catalyst has a palladium content from 0.1 to 10%, in particular 2% or 5%, by weight relative to the total weight of the catalyst,
and in which the surface of the said catalyst, analysed by XPS, comprises from 90 to 100% palladium in the oxidation state (II), in particular in the form of Pd—O or a PdₓCe₁₋ₓO₂ solid solution, x varying from 0.01 to 1.

The surface species, in particular the nature of the bonds with the palladium atoms, and the surface of the catalyst according to the invention can be analysed by spectroscopic techniques such as XPS.

The inventors have surprisingly observed that the palladium atoms on the surface are in the form of palladium with an oxidation state (II) from 90 to 100%, in particular in the form of Pd—O or a PdₓCe₁₋ₓO₂ solid solution, and consequently the absence of metallic palladium with an oxidation state of Pd(0). Although they contain Pd(II), the catalysts according to the invention operate as catalysts for the preparation of oxalates or oxamides by carbonylation from carbon monoxide, an oxidant, an alcohol or an amine respectively. Pd(II) during the reaction is reduced to Pd(0) to form the catalytic active sites (ACS Omega 2018, 3, 11097-11103).

In a particular embodiment, the invention concerns a Pd—X/CeO₂ catalyst as defined above, comprising palladium on a cerium dioxide support,

in which the catalyst has a surface area, analysed by BET, from 100 to 250 m²/g, in particular from 100 to 200 m²/g,
in which the catalyst has a palladium content from 0.1 to 10%, in particular 2% or 5%, by weight relative to the total weight of the catalyst,
in which the catalyst has a fluorine-type structure by XRD and a crystallite size of less than 30 nanometres according to the Scherrer formula, in particular less than 20 nanometres, preferably less than 10 nm
and/or a crystallinity from 0 to 50%, preferably from 0 to 20%.
and/or in which the said catalyst is in the form of particles of average micrometric size, in particular from 1 to 100 μm and/or in which the surface of the said catalyst, analysed by XPS, comprises from 90 to 100% palladium in the oxidation state (II), in particular in the form of Pd—O or a PdₓCe₁₋ₓO₂ solid solution, x varying from 0.01 to 1.

### Support Used to Prepare the Catalyst

In a particular embodiment, the invention concerns a Pd—X/CeO₂ catalyst as defined above, comprising palladium on a cerium dioxide support,

in which the support used to prepare the catalyst has a surface area from 100 to 300 m²/g, in particular from 150 to 160 m²/g or from 270 to 280 m²/g,
and in which the catalyst has a palladium content from 0.1 to 10%, in particular 2% or 5%, by weight relative to the total weight of the catalyst.

According to a particular embodiment, the invention concerns a catalyst defined above, in which the support used for preparing the catalyst has, after calcination at a temperature ranging from 800 to 900° C. for a period from 2 h to 5 h, a surface area from 40 to 60 m²/g, in particular from 45 to 55 m²/g.

According to a particular embodiment, the invention relates to a catalyst as defined above, in which said support used for the preparation of the catalyst has a median pore size (D50) of from 5 to 20 μm, in particular from 6 to 12 μm.

According to a particular embodiment, the invention relates to a catalyst as defined above, in which said support used for the preparation of the catalyst has a loss on ignition (LOI) of less than 8%.

The supports used to prepare the catalyst can be products marketed by Solvay such as HSA 85 and HSA 20SP.

### Dopant

According to a particular embodiment, the invention concerns a Pd—X/CeO₂ catalyst as defined above, said catalyst further comprising a dopant.

According to a particular embodiment, the dopant is selected from the following group of elements: Mn, Mg, Ca, Fe, Ba, Sr, Y, Nb, Zn, Bi, Sn, La, Pr, Nb and Sm.

In a particular embodiment, the dopant is Mn.

According to a particular embodiment, the invention concerns a Pd—X/CeO₂ catalyst as defined above, said catalyst comprising a dopant content from 0.5 to 10%, in particular 1%, by weight relative to the total weight of the catalyst.

According to a particular embodiment, the invention relates to a palladium/cerium dioxide catalyst, comprising palladium on a cerium dioxide support, of formula Pd—X/CeO₂, in which X represents the empty set or a doping element,
in which the catalyst has a surface area, analysed by BET, from 50 to 250 m²/g, in particular from 100 to 200 m²/g,
and in which the catalyst has a palladium content from 0.1 to 10%, in particular 2% or 5%, by weight relative to the total weight of the catalyst,
optionally the dopant being selected from the following group of elements: Mn, Mg, Ca, Fe, Ba, Sr, Y, Nb, Zn, Bi, Sn, La, Pr, Nb and Sm, preferably the dopant being Mn, in particular the said catalyst comprising a dopant content from 0.5 to 10%, in particular 1%, by weight relative to the total weight of the catalyst.

According to a particular embodiment, the invention relates to a palladium/cerium dioxide catalyst, comprising palladium on a cerium dioxide support, of formula Pd—X/CeO₂, in which X represents the empty set or a doping element,
in which the catalyst has a surface area, analysed by BET, from 100 to 250 m²/g, in particular from 100 to 200 m²/g,
and in which the catalyst has a palladium content from 0.1 to 10%, in particular 2% or 5%, by weight relative to the total weight of the catalyst,
optionally the dopant being selected from the following group of elements: Mn, Mg, Ca, Fe, Ba, Sr, Y, Nb, Zn, Bi, Sn, La, Pr, Nb and Sm, preferably the dopant being Mn, in particular the said catalyst comprising a dopant content of from 0.5 to 10%, in particular 1%, by weight relative to the total weight of the catalyst.

According to a particular embodiment, the invention relates to a palladium/cerium dioxide catalyst, comprising palladium on a cerium dioxide support, of formula Pd—X/CeO₂, in which X represents the empty set or a doping element,

in which the catalyst has a surface area, analysed by BET, from 100 to 250 m²/g, in particular from 100 to 200 m²/g, in which the catalyst has a palladium content from 0.1 to 10%, in particular 2% or 5%, by weight relative to the total weight of the catalyst,
in which the catalyst has a fluorine-type structure by XRD and a crystallite size of less than 30 nanometres according to the Scherrer formula, in particular less than 20 nanometres, preferably less than 10 nanometres,
and/or a crystallinity of 0 to 50%, preferably 0 to 20%,
and/or in which said catalyst is in the form of particles of average micrometric size, in particular from 1 to 100 μm,
and/or in which the surface of the said catalyst, analysed by XPS, comprises from 90 to 100% palladium in the oxidation state (II), in particular in the form of Pd—O or a PdₓCe₁₋ₓO₂ solid solution, x varying from 0.01 to 1,
optionally the dopant being selected from the following group of elements: Mn, Mg, Ca, Fe, Ba, Sr, Y, Nb, Zn, Bi, Sn, La, Pr, Nb and Sm, preferably the dopant being Mn, in particular the said catalyst comprising a dopant content from 0.5 to 10%, in particular 1%, by weight relative to the total weight of the catalyst.

### Method for Preparing the Catalyst

Another object of the invention relates to a method for preparing a Pd—X/CeO₂ catalyst, comprising palladium on a CeO₂ support, as defined above, in which the method comprises:
a step D of impregnating a palladium salt, in particular palladium nitrate, on a cerium dioxide support, to obtain a homogeneous material,
a step E for drying the material,
a palladium activation step F, to obtain said catalyst.

According to a particular embodiment, the invention relates to a method for the preparation as defined above of a Pd—X/CeO₂ catalyst, in which step D comprises the use of a support having a surface area of from 100 to 300 m²/g, in particular from 150 to 160 m²/g or from 270 to 280 m²/g.

According to a particular embodiment, the invention relates to a method for preparing as defined above a Pd—X/CeO₂ catalyst, in which step D comprises using a palladium salt concentration calculated to obtain a palladium content from 0.1 to 10%, by weight relative to the total weight of the catalyst.

According to a particular embodiment, the invention relates to a method for preparing a Pd—X/CeO₂ catalyst as defined above, in which step D comprises using a concentration of palladium salt, in particular palladium nitrate, in an amount from 25 to 3000 mg per 10 g of CeO₂ support in order to obtain a palladium content from 0.1 to 10% by weight relative to the total weight of the catalyst.

According to a particular embodiment, the invention relates to a method for preparing as defined above a Pd—X/CeO₂ catalyst, in which step D comprises:
the use of a substrate with a surface area from 100 and 300 m²/g, in particular from 150 and 160 m²/g or from 270 and 280 m²/g,
and using a palladium salt concentration calculated to obtain a palladium content of 0.1 to 10%, by weight relative to the total weight of the catalyst.

According to a particular embodiment, the invention relates to a method for preparing as defined above a Pd—X/CeO₂ catalyst, in which step D additionally comprises at least one other salt chosen from precursors of dopants from the following group of elements: Mn, Mg, Ca, Fe, Ba, Sr, Y, Nb, Zn, Bi, Sn, La, Pr, Nb and Sm.

In a particular embodiment, the salt is a manganese salt.

In a particular embodiment, the dopant salt concentration is calculated to give a dopant content of 0.5 to 10% by weight relative to the total weight of the catalyst.

According to a particular embodiment, the invention relates to a method for preparing as defined above a Pd—X/CeO₂ catalyst, in which step E comprises drying at a temperature from 60° C. to 100° C., in particular 80° C., preferably for a period from 10 to 24 hours, in particular 16 hours.

According to a particular embodiment, the invention relates to a method for preparing as defined above a Pd—X/CeO₂ catalyst, in which step F comprises calcination at a temperature from 200 to 1000° C., in particular 600° C., preferably for a period of 1 to 15 hours, in particular 2 hours.

The expression “from 200 to 1000° C.” corresponds to the following ranges: from 200 to 300° C.; from 300 to 400° C.; from 400 to 500° C.; from 500 to 600° C.; from 600 to 700° C.; from 700 to 800° C.; from 800 to 900° C.; from 900 to 1000° C.

According to a particular embodiment, the invention relates to a method for preparing a Pd—X/CeO₂ catalyst according to the invention as defined above, in which the method comprises:
a step D of impregnating a palladium salt, in particular palladium nitrate, on a cerium dioxide support, to obtain a homogeneous material, in particular said step D comprises:
   the use of a substrate with a surface area from 100 to 300 m²/g, in particular from 150 to 160 m²/g or from 270 to 280 m²/g,
   and using a palladium salt concentration calculated to obtain a palladium content from 0.1 to 10%, by weight relative to the total weight of the catalyst,
   optionally using another salt selected from precursors of dopants from the following group of elements: Mn, Mg, Ca, Fe, Ba, Sr, Y, Nb, Zn, Bi, Sn, La, Pr, Nb and Sm, in particular the manganese salt, the dopant salt concentration is calculated to obtain a dopant content of 0.5 to 10% by weight relative to the total weight of the catalyst,
a step E of drying the material, in particular at a temperature from 60 to 100° C., in particular 80° C., preferably for a period of 10 to 24 hours, in particular 16 hours,
a palladium activation step F, in particular comprising calcination at a temperature from 200 to 1000° C., in particular 600° C., preferably for a period from 1 to 15 hours, in particular 2 hours, to obtain said catalyst.

The following examples and figures illustrate the invention, without limiting its scope.

### FIGURES

FIG. 1 shows the diffractograms obtained from 10 to 90 degrees at 2θ, FIG. 1a) is an X-ray diffractogram of the HSA 20SP ceria support, FIG. 1b) is a diffractogram of the Pd (2%)/CeO₂ catalyst (HSA 20 SP), FIG. 1c) is a diffractogram of the Pd (2%)—Mn (1%)/CeO₂ catalyst (HSA 20 SP).

FIG. 2 shows SEM images of the Pd (2%)/CeO₂ catalyst (HSA 85 SP).

FIG. 3 shows SEM images of the Pd (2%)/CeO₂ catalyst (HSA 20 SP).

FIG. 4 shows SEM images of the Pd (2%)—Mn (1%)/CeO catalyst (HSA 20 SP).

FIG. 5 shows the spectra of the Pd (2%)/CeO₂ catalyst (HSA 20 SP) at the binding energies corresponding to Pd 3d (FIG. 5a), Ce 3d (FIG. 5b) and O 1s (FIG. 5c).

FIG. 6 shows the spectrum of the Pd (2%)/CeO₂ catalyst (HSA 20 SP) at the binding energies corresponding to Pd 3d and the assignment of the corresponding bonds to the peaks.

FIG. 7 shows the spectra of the Pd (2%)/CeO₂ catalyst (HSA 20 SP) at the binding energies corresponding to Pd 3d at two different exposure times.

FIG. 8 shows the spectra of the Pd (2%)—Mn (1%)/CeO₂ catalyst (HSA 20 SP) at the binding energies corresponding to Pd 3d (FIG. 8a), Mn 2p³ (FIG. 8b), Ce 3d (FIG. 8c) and O 1s (FIG. 8d).

### EXAMPLES

### Example 1—Materials and Methods

The two supports used in CeO₂, under the trade names “HSA 85” and “HSA 20 SP” respectively, come from Solvay.

“HSA 85 has a specific surface area of 273 m²/g, a surface area after calcination at 800° C. for 2 hours of 55.0 m²/g, a median pore size D (50) of 6.7 μm and a loss on ignition (LOI) of less than 7.9%.

“HSA 20SP has a specific surface area of 159 m²/g, a surface area after calcination at 900° C. for 5 hours of 45.9 m²/g, a median pore size D (50) of 12 μm and a loss on ignition (LOI) of less than 3%.

The α-Al₂O₃ support was supplied by Sigma Aldrich.

The γ-Al₂O₃, ZrO₂ support and the Pd/C catalyst were supplied by Strem Chemicals (15 Rue de l'Atome, 67800 Bischheim).

Palladium nitrate (Pd(NO₃)₂·xH₂O) and other metal salts (dopants) such as Mn(OAc)₂, Ca(NO₃)₂·3H₂O, Fe(NO₃)₃·9H₂O, Mg(NO₃)₃·6H₂O were supplied by Fischer. The autoclave is supplied by Parr Instrument Company.

### Example 2—General Procedure for the Preparation of Heterogeneous Pd/CeO₂ Catalysts

The palladium salt, Pd(NO₃)₂·xH₂O (corresponding concentration in Pd content by weight with respect to the total weight of the catalyst) was dissolved in a minimum volume of demineralised water, forming a solution. This solution was added to the appropriate amount of cerium dioxide support (HSA 85 or HSA 20 SP) with a solution mass/support mass ratio from 0.6 to 1, and the resulting paste of CeO₂ was mixed at room temperature until a homogeneous material was obtained. The material was then dried at 80° C. for 16 hours and calcined at 600° C. for 2 hours to obtain the catalyst.

### Example 3—Preparation of Heterogeneous Pd/CeO₂ Catalysts and Surface Analysis

Table 1 below shows the conditions for preparing Pd/CeO₂ catalysts according to Example 2.

TABLE 1
Prepared Pd/CeO₂ catalysts

| | CeO₂ Support | | |
|---|---|---|---|
| Catalyst | (g) | Pd(NO₃)₂•xHO₂ | |
| Pd (2%)/CeO₂ | HSA 85 | 504 | mg |
| (HSA 85) | (10 g) | | |
| Pd (5%)/CeO₂ | HSA 85 | 1260 | mg |
| (HSA 85) | (10 g) | | |
| Pd (1%)/CeO₂ | HSA 20 SP | 257 | mg |
| (HSA 20SP) | (10 g) | | |
| Pd (2%)/CeO₂ | HSA 20 SP | 504 | mg |
| (HSA 20SP) | (10 g) | | |
| Pd (5%)/CeO₂ | HSA 20 SP | 1260 | mg |
| (HSA 20SP) | (10 g) | | |
| Pd (10%)/CeO₂ | HSA 20 SP | 2570 | mg |
| (HSA 20SP) | (10 g) | | |

Table 2 shows the results of the analysis of the specific surface area of the catalysts prepared by the BET method.

TABLE 2
Surface area of prepared Pd/CeO₂ catalysts

| | Specific surface |
|---|---|
| Catalysts | area (m²/g) |
| Pd (2%)/CeO₂ (HSA 85) | 185.79 m²/g |
| Pd (2%)/CeO₂ (HSA 20SP) | 142.15 m²/g |

### Example 4—General Procedure for the Preparation of Heterogeneous Pd-M/CeO₂ Catalysts

Pd(NO₃)₂·xH₂O and the metal salt (corresponding dopant) were dissolved in a minimum volume of demineralised water, forming a solution. This solution containing the metal precursors was added to the appropriate amount of cerium dioxide support (HSA 85 or HSA 20 SP) and the resulting paste of CeO₂ was mixed at room temperature until a homogeneous material was obtained. The material was then dried at 80° C. for 16 hours and calcined at 600° C. for 2 hours to obtain the catalyst.

### Example 5—Preparation of Heterogeneous Pd—X/CeO₂-Doped Catalysts₂ and Analysis

Table 3 below shows the preparation conditions for the Pd/CeO₂ catalysts prepared according to example 4.

TABLE 3
Prepared Pd-X/CeO₂ doped catalysts

| | CeO₂ | | Salt of |
|---|---|---|---|
| | Support | | the dopant |
| Catalyst | (g) | Pd(NO₃)₂•xHO₂ | (mg) |
| Pd(2%)-Mn(0.5%)/ | HSA 20 SP | 504 mg | Mn(OAc)₂ |
| CeO₂ | (10 g) | | (165 mg) |
| (HSA 20 SP) | | | |
| Pd (2%)-Mn(1%)/ | HSA 20 SP | 504 mg | Mn(OAc)₂ |
| CeO₂) | (10 g) | | (330 mg) |
| (HSA 20 SP) | | | |
| Pd (2%)-Mn(2%)/ | HSA 20 SP | 504 mg | Mn(OAc)₂ |
| CeO₂ | (10 g) | | (660 mg) |
| (HSA 20 SP) | | | |
| Pd (2%)-Mn(5%)/ | HSA 20 SP | 504 mg | Mn(OAc)₂ |
| CeO₂ | (10 g) | | (825 mg) |
| (HSA 20 SP) | | | |
| Pd(2%)-Fe(1%)/ | HSA 20 SP | 504 mg | Fe(NO₃)₃•9H O₂ |
| CeO₂ | (10 g) | | (769 mg) |
| (HSA 20 SP) | | | |
| Pd(2%)-Mg(1%)/ | HSA 20 SP | 504 mg | Mg(NO₃)₃•6H O₂ |
| CeO₂ | (10 g) | | (1,310 mg) |
| (HSA 20 SP) | | | |
| Pd (2%)-Ca(1%)/ | HSA 20 SP | 504 mg | Ca(NO₃)₂•3H O₂ |
| CeO₂ | (10 g) | | (699 mg) |
| (HSA 20 SP) | | | |

Table 4 shows the results of the BET surface area analysis of a Pd—X-doped catalyst.

TABLE 4
Surface area of the prepared Pd-Mn/CeO₂ catalyst

| | Specific surface |
|---|---|
| Catalysts | area (m²/g) |
| Pd (2%)Mn(1%)/CeO₂ | 132.10 m²/g |
| (HSA 20SP) | |

### Example 6-Preparation of Heterogeneous Pd Catalysts on Other Supports

Pd/γ-Al₂O₃ and Pd/ZrO₂ Catalysts

The palladium salt, Pd(NO₃)₂·xH₂O (corresponding concentration in terms of Pd content by weight relative to the total weight of the catalyst) was dissolved in a minimum volume of demineralised water, forming a solution. The solution was added to the appropriate amount of oxide support (γ-A Al₂O₃ or ZrO₂); the resulting paste was mixed at room temperature until a homogeneous mixture was obtained. The material was then dried at 80° C. for 16 h. The catalyst was then calcined at 600° C. for 2 hours.

Table 5 below shows the preparation conditions for the Pd/γ-Al O₂₃ and Pd/ZrO₂. catalysts.

TABLE 5
Prior art catalysts

| Catalyst | Support (g) | Pd(NO₃)₂•xH₂O |
|---|---|---|
| Pd (2%)/γ- Al₂O₃ | γ- Al₂O₃ (10 g) | 504 mg |
| Pd (5%)/ZrO₂ | ZrO₂ (10 g) | 504 mg |

Pd/C catalysts: The Pd (10%)/C catalyst is supplied by Strem.
PdCl₂/CeO₂: catalyst The PdCl₂ (3%)/CeO₂ catalyst is prepared according to the Gaffney et al. paper (Journal of Catalysis, 1984, 90, 261-269).
Pd—Ce/α-Al₂O₃ catalysts: The Pd (1%)—Ce (0.8%)/α-Al₂O₃ catalyst is prepared according to Appl. Cat. A, 2005, 284 (1-2), 253-257.
Pd/CeO₂ catalysts—α-Al₂O₃: The Pd/CeO₂ catalyst—α-Al₂O₃ is prepared according to RSC Adv., 2014, 4, 48901-48904.

### Example 7—General Heterogeneous Catalysis Procedure for the Oxidative Carbonylation of Methanol to Oxalates

A heterogeneous palladium-based catalyst (0.7 mmol or 0.24 mmol Pd), tetrabutylammonium iodide TBAI (554 mg, 1.5 mmol) as promoter, tri-ethylamine Et₃N (0.14 mL, 1.0 mmol), acetonitrile (50 mL) and methanol (25 mL) were introduced into a 450 mL Parr autoclave equipped with a magnetic stirrer. The reactor was sealed and the reaction mixture purged three times with nitrogen (5 bars) and twice with oxygen (5 bars).

The autoclave was then pressurised with 15 bars of oxygen and a further 65 bars of carbon monoxide (total pressure of 80 bars). The reaction medium was then stirred at 90° C. for 16 h or 60 h.

Once the reaction was complete, the autoclave was brought to room temperature before being depressurised and purged three times with nitrogen (5 bars).

The final mixture obtained was then filtered and transferred to a 250 mL flask.

The reaction solvent and excess alcohol were separated by evaporation on a rotary evaporator. The dimethyloxalate was recovered after purification by recrystallisation in di-ethyl ether and the isolated yields were calculated.

For reactions with alcohols, the results are described in terms of NCC rather than percentage yield due to the excess use of the substrate, the alcohol (or also the absence of solvent, in this case substrate=solvent).

The NCC is calculated as follows:

NCC=number of moles of product formed/number of moles of Pd

### Example 8: Reaction with 0.7 Mmol of Catalyst

Table 6 below shows the conditions for preparing dimethyloxalate with Pd/CeO₂ catalysts and Pd on other supports (for comparison) and the yield results obtained in terms of isolated mass and NCC.

TABLE 6
Conditions for the preparation of dimethyloxalate with heterogeneous palladium
catalysts at a content of 0.7 mmol and the results obtained.

| | | Base | Promoteur | Solvent, | O₂/CO | T | Time, | | |
|---|---|---|---|---|---|---|---|---|---|
| Reaction | Cat | (mmol) | (mmol) | (ml) | (bar) | (° C.) | (h) | Substrate | Results |
| M1 | Pd(2%)- | Et₃N | TBAI | MeCN; | 15/65 | 90 | 60 | 25 mL | 7.41 g |
| | CeO₂ | (1 | (1.5 mmol) | 50 mL | | | | MeOH | NCC: |
| | (HSA 20 | mmol) | | | | | | | 90 |
| | SP) | | | | | | | | |
| | (0.7 mmol) | | | | | | | | |
| M2 | Pd(5%)- | Et₃N | TBAI | MeCN; | 15/65 | 90 | 60 | 25 mL | 6.6 g |
| | CeO₂ | (1 | (1.5 mmol) | 50 mL | | | | MeOH | NCC: |
| | (HSA 85) | mmol) | | | | | | | 80 |
| | (0.7 mmol) | | | | | | | | |
| M3 | Pd(5%)- | Et₃N | TBAI | MeCN; | 15/65 | 90 | 60 | 25 mL | 6.35 g |
| | CeO₂ | (1 | (1.5 mmol) | 50 mL | | | | MeOH | NCC: |
| | (HSA 20 | mmol) | | | | | | | 77 |
| | SP) | | | | | | | | |
| | (0.7 mmol) | | | | | | | | |
| M4 | Pd(5%)-γ- | Et₃N | TBAI | MeCN; | 15/65 | 90 | 16 | 25 mL | 3 g |
| | Al₂O₃ | (1 | (1.5 mmol) | 50 mL | | | | MeOH | NCC: |
| | (0.7 mmol) | mmol) | | | | | | | 36 |
| M5 | Pd(5%)-γ- | Et₃N | TBAI | MeCN; | 15/65 | 90 | 60 | 25 mL | 3.5 g |
| | Al₂O₃ | (1 | (1.5 mmol) | 50 mL | | | | MeOH | NCC: |
| | (0.7 mmol) | mmol) | | | | | | | 42 |
| M6 | Pd(10%)/C | Et₃N | TBAI | MeCN; | 15/65 | 90 | 16 | 25 mL | 5.7 g |
| | (0.7 mmol) | (1 | (1.5 mmol) | 50 mL | | | | MeOH | NCC: |
| | | mmol) | | | | | | | 69 |

Carried out under the same conditions, the results of M1, M2 and M3 with the catalysts of the invention show a higher yield by mass of isolated product and a higher NCC than those of the tests with the catalysts on different supports of the prior art (M4, M5 and M6). The yield of the catalysts according to the invention is greater than 6 g. In particular, M1 shows that a preparation with a catalyst containing 2% Pd on the HSA 20 SP support gives a higher yield with catalysts containing 5% Pd.

Under the same operating conditions, the results of the invention's Pd/CeO₂ catalysts are superior to those of the prior art.

Furthermore, in this range of 100 to 250 m²/g, the catalysts were unexpectedly found to have substantially equivalent activities, as demonstrated by the tests in Table 6. A comparison of tests M2 and M3 shows that the Pd (5%)/HSA 85 catalyst (186 m²/g) and the Pd (5%)/HSA 20 SP catalyst (142 m²/g), i.e. with a difference of approximately 44 m²/g, exhibit almost identical yields with NCCs of 80 and 77 respectively.

### Example 9:0.24 Mmol Catalyst

Table 7 below shows the conditions under which dimethyloxalate was prepared using Pd/CeO₂ catalysts at a content of 0.24 mmol and Pd catalysts on other supports (for comparison) and the yield results obtained in terms of product mass and NCC.

TABLE 7
Conditions for the preparation of dimethyloxalate with heterogeneous
palladium catalysts at a content of 0.24 mmol and results obtained.

| | | Base | Promoteur | Solvent, | O₂/CO | T | Time, | | |
|---|---|---|---|---|---|---|---|---|---|
| Reaction | Cat | (mmol) | (mmol) | (ml) | (bar) | (° C.) | (h) | Substrate | Results |
| M7 | Pd(1%)- | Et₃N | TBAI | MeCN; | 15/65 | 90 | 16 | 25 mL | 3.32 g |
| | CeO₂ | (1 | (1.5 mmol) | 50 mL | | | | MeOH | NCC: |
| | (HSA 20 | mmol) | | | | | | | 117 |
| | SP) | | | | | | | | |
| | (0.24 | | | | | | | | |
| | mmol) | | | | | | | | |
| M8 | Pd(2%)- | Et₃N | TBAI | MeCN; | 15/65 | 90 | 16 | 25 mL | 4.77 g |
| | CeO₂ | (1 | (1.5 mmol) | 50 mL | | | | MeOH | NCC: |
| | (HSA 20 | mmol) | | | | | | | 168 |
| | SP) | | | | | | | | |
| | (0.24 | | | | | | | | |
| | mmol) | | | | | | | | |
| M9 | Pd(5%)- | Et₃N | TBAI | MeCN; | 15/65 | 90 | 16 | 25 mL | 3 g |
| | CeO₂ | (1 | (1.5 mmol) | 50 mL | | | | MeOH | NCC: |
| | (HSA 20 | mmol) | | | | | | | 106 |
| | SP) | | | | | | | | |
| | (0.24 | | | | | | | | |
| | mmol) | | | | | | | | |
| M10 | Pd(10%)- | Et₃N | TBAI | MeCN; | 15/65 | 90 | 16 | 25 mL | 3.7 g |
| | CeO₂ | (1 | (1.5 mmol) | 50 mL | | | | MeOH | NCC: |
| | (HSA 20 | mmol) | | | | | | | 130 |
| | SP) | | | | | | | | |
| | (0.24 | | | | | | | | |
| | mmol) | | | | | | | | |
| M11 | Pd(2%)- | Et₃N | TBAI | MeCN; | 15/65 | 90 | 16 | 25 mL | 1.77 g |
| | □-Al₂O₃ | (1 | (1.5 mmol) | 50 mL | | | | MeOH | NCC: |
| | (0.24 | mmol) | | | | | | | 63 |
| | mmol) | | | | | | | | |
| M12 | Pd(2%)- | Et₃N | TBAI | MeCN; | 15/65 | 90 | 16 | 25 mL | 1.81 g |
| | □-Al₂O₃ | (1 | (1.5 mmol) | 50 mL | | | | MeOH | NCC: |
| | (0.24 | mmol) | | | | | | | 64 |
| | mmol) | | | | | | | | |
| M13 | Pd(2%)- | Et₃N | TBAI | MeCN; | 15/65 | 90 | 16 | 25 mL | 3.2 g |
| | □r□_{□}(0.24 | (1 | (1.5 mmol) | 50 mL | | | | MeOH | NCC: |
| | mmol) | mmol) | | | | | | | 113 |
| M14 | Pd(10%/C) | Et₃N | TBAI | MeCN; | 15/65 | 90 | 16 | 25 mL | 4.4 g |
| | (0.24 | (1 | (1.5 mmol) | 50 mL | | | | MeOH | NCC: |
| | mmol) | mmol) | | | | | | | 155 |

At a similar Pd content of 2%, the results of M8 with a catalyst according to the invention are superior to the results of Pd catalysts on different oxide supports (M11, M12 and M13).

The results of M7, M8, M9 and M10 on Pd/CeO₂ catalysts suggest that optimisation by Pd content is possible.

Tests M8 and M14 indicate that the Pd/CeO₂ catalyst can perform better than a commercial carbon-supported Pd catalyst.

### Example 10: Reaction with a Prior Art Catalyst

Table 8 below shows the preparation conditions with prior art Pd catalysts (for comparison) according to example 6 and the yields obtained.

TABLE 8
Conditions for the preparation of dimethyloxalate and results obtained with
the heterogeneous palladium catalysts of the prior art on different supports.

| | | Base | Promoteur | Solvent, | O₂/CO | T | Time, | | |
|---|---|---|---|---|---|---|---|---|---|
| Reaction | Cat | (mmol) | (mmol) | (ml) | (bar) | (° C.) | (h) | Substrate | Results |
| A1 | Pd(3%)Cl₂/ | Et₃N | TBAI | MeCN; | 15/65 | 90 | 16 | 25 mL | 0.750 g |
| | CeO₂ | (1 | (1.5 mmol) | 50 mL | | | | MeOH | NCC: |
| | (0.24 | mmol) | | | | | | | 26 |
| | mmol) | | | | | | | | |
| A2 | Pd(1%)- | Et₃N | TBAI | MeCN; | 15/65 | 90 | 60 | 25 mL | 3.12 g |
| | Ce (0.8%)/ | (1 | (1.5 mmol) | 50 mL | | | | MeOH | NCC: |
| | □-Al₂O₃ | mmol) | | | | | | | 110 |
| | (0.24 | | | | | | | | |
| | mmol) | | | | | | | | |
| A3 | Pd/CeO₂- | Et₃N | TBAI | MeCN; | 15/65 | 90 | 60 | 25 mL | 1.00 g |
| | □-Al₂O₃ | (1 | (1.5 mmol) | 50 mL | | | | MeOH | NCC: |
| | (0.24 | mmol) | | | | | | | 35 |
| | mmol) | | | | | | | | |

Comparison of the tests with a catalyst according to the invention (M8) and test A1, i.e. a catalyst prepared according to Gaffney et al. reveals an NCC yield of 168 for M8 and a lower NCC yield of 26 for A1, which confirms the superior efficiency of the catalysts according to the invention.

### Example 11: Reaction with a Pd-M/CeO₂ Bimetallic Catalyst

Table 9 below shows the preparation conditions and results with Pd—X-doped catalysts.

TABLE 9
Preparation conditions for dimethyloxalates and results
obtained with Pd-X/CeO doped heterogeneous catalysts₂.

| | | Base | Promoteur | Solvent, | O₂/CO | T | Time, | | |
|---|---|---|---|---|---|---|---|---|---|
| Reaction | Cat | (mmol) | (mmol) | (ml) | (bar) | (C) | (h) | Substrate | Results |
| D1 | Pd(2%)- | Et₃N | TBAI | MeCN; | 15/65 | 90 | 16 | 25 mL | 4.97 g |
| | Mn(1%)/ | (1 | (1.5 mmol) | 50 mL | | | | MeOH | NCC: |
| | CeO₂ | mmol) | | | | | | | 176 |
| | (HSA | | | | | | | | |
| | 20 SP) | | | | | | | | |
| | (0.24 | | | | | | | | |
| | mmol) | | | | | | | | |
| D2 | Pd(2%)- | Et₃N | TBAI | MeCN; | 15/65 | 90 | 16 | 25 mL | 2.52 g |
| | Ca(1%)- | (1 | (1.5 mmol) | 50 mL | | | | MeOH | NCC: |
| | CeO₂ | mmol) | | | | | | | 89 |
| | (HSA | | | | | | | | |
| | 20 SP) | | | | | | | | |
| | (0.24 | | | | | | | | |
| | mmol) | | | | | | | | |

Tests D1 and D2 show the effect of doping the Pd/CeOz catalyst.

In particular, manganese doping improves the properties of the catalyst.

### Example 12: Reaction with Pd—Mn/CeO₂ Catalyst

Table 10 below shows the conditions under which dimethyloxalate was prepared using Pd—Mn/CeO₂ manganese-doped catalysts and the yield results obtained in terms of product mass and NCC.

TABLE 10
Conditions for preparing dimethyloxalate with manganese-doped
Pd-X/CeO₂ heterogeneous catalysts and results obtained.

| | | Base | Promoteur | Solvent, | O₂/CO | T | Time, | | |
|---|---|---|---|---|---|---|---|---|---|
| Reaction | Cat | (mmol) | (mmol) | (ml) | (bar) | (° C.) | (h) | Substrate | Results |
| M8 | Pd(2%)- | Et₃N | TBAI | MeCN; | 15/65 | 90 | 16 | 25 mL | 4.77 g |
| | CeO₂ | (1 | (1.5 mmol) | 50 mL | | | | MeOH | NCC: |
| | (HSA 20 | mmol) | | | | | | | 168 |
| | SP) (0.24 | | | | | | | | |
| | mmol) | | | | | | | | |
| D3 | Pd(2%)- | Et₃N | TBAI | MeCN; | 15/65 | 90 | 16 | 25 mL | 3.43 g |
| | Mn(0.5%)- | (1 | (1.5 mmol) | 50 mL | | | | MeOH | NCC: |
| | CeO₂ | mmol) | | | | | | | 121 |
| | (HSA 20 | | | | | | | | |
| | SP) (0.24 | | | | | | | | |
| | mmol) | | | | | | | | |
| D1 | Pd(2%)- | Et₃N | TBAI | MeCN; | 15/65 | 90 | 16 | 25 mL | 4.97 g |
| | Mn(1%)- | (1 | (1.5 mmol) | 50 mL | | | | MeOH | NCC: |
| | CeO₂ | mmol) | | | | | | | 176 |
| | (HSA 20 | | | | | | | | |
| | SP) (0.24 | | | | | | | | |
| | mmol) | | | | | | | | |
| D4 | Pd(2%)- | Et₃N | TBAI | MeCN; | 15/65 | 90 | 16 | 25 mL | 3.53 g |
| | Mn(2%)- | (1 | (1.5 mmol) | 50 mL | | | | MeOH | NCC: |
| | CeO₂ | mmol) | | | | | | | 125 |
| | (HSA 20 | | | | | | | | |
| | SP) (0.24 | | | | | | | | |
| | mmol) | | | | | | | | |
| D5 | Pd(2%)- | Et₃N | TBAI | MeCN; | 15/65 | 90 | 16 | 25 mL | 2.68 g |
| | Mn(5%)- | (1 | (1.5 mmol) | 50 mL | | | | MeOH | NCC: |
| | CeO₂ | mmol) | | | | | | | 95 |
| | (HSA 20 | | | | | | | | |
| | SP) (0.24 | | | | | | | | |
| | mmol) | | | | | | | | |

### Example 13—General Heterogeneous Catalysis Procedure for the Oxidative Carbonylation of Ethanol to Oxalates

In a 450 mL Parr autoclave, equipped with a magnetic stirrer, a heterogeneous palladium catalyst (0.7 mmol or 0.24 mmol Pd), tetrabutylammonium iodide TBAI (1.5 to 3.6 mmol), tri-ethylamine Et₃N (1.0 to 4.75 mmol), optionally acetonitrile MeCN (25 to 75 mL) as solvent and ethanol (25 to 75 mL) have been introduced. The reactor was sealed and the reaction mixture was purged three times with nitrogen (5 bars) and twice with oxygen (5 bars).

The autoclave was then pressurised with 15 bars of oxygen and a further 65 bars of carbon monoxide (total pressure of 80 bars). The reaction medium was then stirred at 90° C. for 16 h. Once the reaction was complete, the autoclave was brought to room temperature before being depressurised and purged three times with nitrogen (5 bars).

The final mixture obtained was then filtered and transferred to a 250 mL flask.

The reaction solvent and excess alcohol were separated by evaporation on a rotary evaporator.

The diethyloxalate was recovered after purification by vacuum distillation at 120° C./50-20 mbars and the isolated yields were calculated.

### Example 14: Heterogeneous Catalysis of the Oxidative Carbonylation of Ethanol to Oxalates

Table 11 below shows the preparation conditions using the heterogeneous Pd—X/CeO₂ catalysts for diethyloxalate and the yield results obtained in terms of mass of product isolated and NCC.

TABLE 11
Conditions for the preparation of diethyloxalate with
heterogeneous Pd-X/CeO₂ catalysts and results obtained.

| | | Base | Promoter | Solvent, | O₂/CO | T, | Time, | | |
|---|---|---|---|---|---|---|---|---|---|
| Reaction | Cat | (mmol) | (mmol) | (ml) | (bar) | (° C.) | (h) | Substrate | Results |
| E | Pd(5%)/ | Et₃N | TBAI | x | 15/65 | 90 | 16 | 75 mL | 5.6 g |
| | CeO₂ | (1.5) | (2) | | | | | EtOH | NCC = |
| | (HSA 85) | | | | | | | | 55 |
| | (0.7 | | | | | | | | |
| | mmol) | | | | | | | | |
| E2 | Pd(5%)/CeO₂ | Et₃N | TBAI | MeCN; | 15/65 | 90 | 16 | 50 mL | 7.0 g |
| | (HAS 85) | (1.5) | (2) | 100 mL | | | | EtOH | NCC = |
| | (0.7 | | | | | | | | 68 |
| | mmol) | | | | | | | | |
| E3 | Pd(2%)/CeO₂ | Et₃N | TBAI | MeCN; | 15/65 | 90 | 16 | 25 mL | 5.3 g |
| | (HSA 20 | (1) | (1.5) | 50 mL | | | | EtOH | NCC = |
| | SP) | | | | | | | | 151 |
| | (0.24 | | | | | | | | |
| | mmol) | | | | | | | | |
| E4 | Pd(2%)- | Et₃N | TBAI | MeCN; | 15/65 | 90 | 16 | 25 mL | 6.0 g |
| | Mn(1)- | (1) | (1.5) | 50 mL | | | | EtOH | NCC = |
| | CeO₂ | | | | | | | | 171 |
| | (HSA 20 | | | | | | | | |
| | SP) (0.24 | | | | | | | | |
| | mmol) | | | | | | | | |

Reaction E1 shows that it is possible to operate without a solvent.

### Example 15—Heterogeneous Catalysis Procedure for the Oxidative Carbonylation of Isopropanol to Oxalates

A heterogeneous palladium catalyst (0.7 mmol Pd), tetrabutylammonium iodide (554 mg, 1.0 mmol), triethylamine (1.5 mmol), acetonitrile (50 mL) and isopropanol (25 mL) were added to a 450 mL Parr autoclave equipped with a magnetic stirrer. The reactor was sealed and the reaction mixture purged three times with nitrogen (5 bars) and twice with oxygen (5 bars). The autoclave was then pressurised with 15 bars of oxygen and a further 65 bars of carbon monoxide (total pressure of 80 bars). The reaction medium was then stirred at 90° C. for 16 h. Once the reaction was complete, the autoclave was brought to room temperature before being depressurised and purged three times with nitrogen (5 bars).

The final mixture obtained was then filtered and transferred to a 250 mL flask.

The reaction solvent and excess alcohol were separated by evaporation on a rotary evaporator. The oxalate was recovered after purification (vacuum distillation or recrystallisation) and the isolated yields were calculated.

Table 12 below shows the preparation conditions.

TABLE 12
Conditions for the preparation of diisopropyloxalate
with a heterogeneous Pd-X/CeO₂ catalyst

| | | Base | Promoter | Solvent | O₂/CO | T, | Time, | |
|---|---|---|---|---|---|---|---|---|
| Reaction | Catalyst | (mmol) | (mmol) | (ml) | (bar) | (° C.) | (h) | Substrate |
| P1 | Pd(5%)/CeO₂ | Et₃N | TBAI | MeCN; | 15/65 | 90 | 16 | 25 mL |
| | (HSA | (1) | (1.5) | 50 mL | | | | iPrOH |
| | 85) | | | | | | | |
| | (0.7 | | | | | | | |
| | mmol) | | | | | | | |

### Example 16: General Heterogeneous Catalysis Procedure for the Oxidative Carbonylation of Piperidine to Oxamides

Heterogeneous palladium catalyst (0.7 mmol Pd), tetrabutylammonium iodide TBAI (554 mg, 1.5 mmol), optionally tri-ethylamine Et₃N (0.14 mL, 1.0 mmol) as added base, MeCN acetonitrile (50 mL) and (1.98 mL, 20 mmol) piperidine were introduced into a 450 mL Parr autoclave, equipped with a magnetic stirrer. The reactor was sealed and the reaction mixture purged three times with nitrogen (5 bars) and twice with oxygen (5 bars). The autoclave was then pressurised with 10 bars of oxygen and a further 45 bars of carbon monoxide (total pressure 55 bars). The reaction medium was then kept stirred at 25° C. for 16 h. Once the reaction was complete, the autoclave was depressurised and purged three times with nitrogen (5 bars). The final mixture obtained was then filtered and transferred to a 250 mL flask. The solvent was evaporated and the residue obtained was solubilised in toluene and then filtered over Celite®. The toluene solution was evaporated and a yellow solid was obtained.

### Example 17: Preparation of a Piperidine Oxamide Compound

Table 13 below shows the conditions for preparing oxamide from piperidine using Pd/CeO₂ catalysts and the percentage yield results.

TABLE 13
Conditions for the preparation of oxamide with piperidine and
results obtained with the heterogeneous Pd-X/CeO₂ catalysts.

| | | | Base | Promoter | Solvent, | O₂/CO | T | Time | |
|---|---|---|---|---|---|---|---|---|---|
| Reaction | substrate | Cat | (mmol) | (mmol) | (ml) | (bar) | (° C.) | (h) | Yield |
| Pi1 | 20 mmol | Pd(5%)- | Et₃N | TBAI | MeCN; | 10/45 | 25 | 16 | 95% |
| | | CeO₂ | (1 | (1.5 mmol) | 50 mL | | | | |
| | | (HSA | mmol) | | | | | | |
| | | 85) | | | | | | | |
| | | (0.7 | | | | | | | |
| | | mmol) | | | | | | | |
| Pi2 | 20 mmol | Pd(2%)- | Et₃N | TBAI | MeCN; | 10/45 | 25 | 16 | 94% |
| | | CeO₂ | (1 | (1.5 mmol) | 50 mL | | | | |
| | | (HSA | mmol) | | | | | | |
| | | 20 SP) | | | | | | | |
| | | (0.7 | | | | | | | |
| | | mmol) | | | | | | | |
| Pi3 | 20 mmol | Pd(2%)- | X | TBAI | MeCN; | 10/45 | 25 | 16 | 74% |
| | | CeO₂ | | (1.5 mmol) | 50 mL | | | | |
| | | (HSA | | | | | | | |
| | | 20 SP) | | | | | | | |
| | | (0.7 | | | | | | | |
| | | mmol)) | | | | | | | |

Reactions Pi1, Pi2 and Pi3 were carried out at room temperature, so it was not necessary to heat the reaction medium.

The Pi1 and Pi2 reactions showed yields of over 90%, in the presence of the base, triethylamine. Reaction Pi3 shows that it is possible to operate without adding a base, with the amine acting as the base in the reaction medium.

Example 18 Heterogeneous Palladium-Manganese/CeO₂ catalysis of the oxidative carbonylation of ethanol to oxalates

The Pd (2%)—Mn (1%)/CeO₂ catalyst (HSA 20SP) was prepared according to example 5. The tests were carried out in 2 autoclaves of different volumes:
or in a 450 mL Parr autoclave
or in a 1 L Parr autoclave when 100 mL of the substrate is introduced.

A 450 mL Parr autoclave equipped with a magnetic stirrer was charged with a heterogeneous palladium-based catalyst (0.24 mmol Pd), tetrabutylammonium iodide (554 mg, 1.5 mmol), tri-ethylamine (0.28 mL, 2.0 mmol), acetonitrile (50 mL) and ethanol (50 mL). The reactor was sealed and the reaction mixture was purged three times with nitrogen (5 bars) and twice with oxygen (5 bars). The autoclave was then pressurised with 15 bars of oxygen and a further 65 bars of carbon monoxide (total pressure 80 bars). The reaction mixture was then stirred at 90° C. for 16 h. Once the reaction was complete, the autoclave was returned to room temperature before being depressurised and purged three times with nitrogen (5 bars). The reaction mixture was then filtered and the solution transferred to a 250 mL flask. The reaction solvent and excess alcohol were separated by evaporation on a rotary evaporator. The oxalate was recovered after purification (vacuum distillation at 120° C./50-20 mbars for the diethyloxalate) and the isolated yields were calculated.

A 1 L Parr autoclave equipped with a magnetic stirrer was charged with a heterogeneous palladium-based catalyst (0.48 mmol Pd), tetrabutylammonium iodide (1.11 g, 3 mmol), tri-ethylamine (0.56 mL, 4.0 mmol), acetonitrile (100 mL) and ethanol (100 mL). The reactor was sealed and the reaction mixture was purged three times with nitrogen (5 bars) and twice with oxygen (5 bars). The autoclave was then pressurised with 15 bars of oxygen and a further 65 bars of carbon monoxide (total pressure 80 bars). The reaction mixture was then stirred at 90° C. for 16 h. Once the reaction was complete, the autoclave was returned to room temperature before being depressurised and purged three times with nitrogen (5 bars). The reaction mixture was then filtered and the solution transferred to a 500 mL flask. The reaction solvent and excess alcohol were separated by evaporation on a rotary evaporator. The oxalate was recovered after purification (vacuum distillation at 120° C./50-20 mbars for the diethyloxalate) and the isolated yields were calculated.

Table 14 below shows the conditions under which diethyloxalate was prepared using Pd (2%)—Mn(1%)/CeO₂ catalysts and the results obtained in terms of product mass and NCC.

TABLE 14
Conditions for the preparation of diethyloxalate with Pd(2%)-Mn(1%)/CeO
catalysts₂ and the results obtained in terms of product mass and NCC.

| | | Base | Promoter | Solvant | O2/CO | T | Time | | |
|---|---|---|---|---|---|---|---|---|---|
| Reaction | Cat | (mmol) | (mmol) | (ml) | (bar) | ° C. | (h) | Substrate | Results |
| 1 | Pd(2%)- | Et3N | TBAI | MeCN; | 15/65 | 90 | 16 | 25 mL | 6 g |
| | Mn(1%)-CeO2 | (1) | (1.5) | 50 mL | | | | EtOH | NCC = |
| | (HSA 20 SP) | | | | | | | | 171 |
| | (0.24 mmol) | | | | | | | | |
| 2 | Pd(2%)- | Et3N | TBAI | MeCN; | 15/65 | 90 | 16 | 50 mL | 8.5 g |
| | Mn(1%)-CeO2 | (1) | (1.5) | 50 mL | | | | EtOH | NCC = |
| | (HSA 20 SP) | | | | | | | | 242 |
| | (0.24 mmol) | | | | | | | | |
| 3 | Pd(2%)- | Et3N | TBAI | MeCN; | 15/65 | 90 | 16 | 75 mL | 6.2 g |
| | Mn(1%)-CeO2 | (1) | (1.5) | 25 mL | | | | EtOH | NCC = |
| | (HSA 20 SP) | | | | | | | | 177 |
| | (0.24 mmol) | | | | | | | | |
| 4 | Pd(2%)- | Et3N | TBAI | MeCN; | 15/65 | 90 | 16 | 100 mL | 18.7 g |
| | Mn(1%)-CeO2 | (2) | (3) | 100 mL | | | | EtOH | NCC = |
| | (HSA 20 SP) | | | | | | | | 267 |
| | (0.48 mmol) | | | | | | | | (reactor |
| | | | | | | | | | 1 L) |
| 5 | Pd(2%)- | Et3N | NaI | MeCN | 15/65 | 90 | 16 | 50 mL | 1.88 g |
| | Mn(1%)-CeO2 | (1) | (1.5) | 50 mL | | | | EtOH | NCC = |
| | (HSA 20 SP) | | | | | | | | 54 |
| | (0.24 mmol) | | | | | | | | |
| 6 | Pd(2%)- | Et3N | TBAI | MeCN | 15/65 | 90 | 16 | 50 mL | 9.58 g |
| | Mn(1%)-CeO2 | (2) | (1.5) | 50 mL | | | | EtOH | NCC = |
| | (HSA 20 SP) | | | | | | | | 273 |
| | (0.24 mmol) | | | | | | | | |
| 7 | Pd(2%)- | Et3N | TBAI | MeCN | 15/65 | 90 | 16 | 50 mL | 11 g |
| | Mn(1%)-CeO2 | (2) | (1.5) | 50 mL | | | | EtOH | NCC = |
| | (HSA 20 SP) | | | | | | | | 157 |
| | (0.48 mmol) | | | | | | | | |
| 8 | Pd(2%)- | Et3N | TBAI | MeCN | 15/65 | 90 | 16 | 50 mL | 0.48 g |
| | Mn(1%)-CeO2 | (0) | (1.5) | 50 mL | | | | EtOH | NCC = |
| | (HSA 20 SP) | | | | | | | | 14 |
| | (0.24 mmol) | | | | | | | | |
| 9 | Pd(2%)- | Et3N | TBAI | MeCN | 10/45 | 90 | 16 | 50 mL | 5.94 g |
| | Mn(1%)-CeO2 | (1) | (1.5) | 50 mL | | | | EtOH | NCC = |
| | (HSA 20 SP) | | | | | | | | 170 |
| | (0.24 mmol) | | | | | | | | |
| 10 | Pd(2%)- | Et3N | TBAI | MeCN | 15/65 | 90 | 16 | 50 mL | 7.5 g |
| | Mn(1%)-CeO2 | (4) | (1.5) | 50 mL | | | | EtOH | NCC = |
| | (HSA 20 SP) | | | | | | | | 214 |
| | (0.24 mmol) | | | | | | | | |
| 11 | Pd(2%)- | Et3N | TBAI | MeCN | 15/65 | 90 | 16 | 100 mL | 23 g |
| | Mn(1%)-CeO2 | (4) | (3) | 100 mL | | | | EtOH | NCC = |
| | (HSA 20 SP) | | | | | | | | 328 |
| | (0.24 mmol) | | | | | | | | (reactor |
| | | | | | | | | | 1 L) |

### Example 19: Structural Characterisation of Catalysts

X-ray powder diffraction analysis was carried out on the HSA 20SP ceria support, the Pd (2%)/CeO₂ catalyst (HSA 20 SP) prepared according to example 3 and the Pd (2%)—Mn(1%)/CeO₂ catalyst (HSA 20 SP) prepared according to example 5 were analysed by X-ray diffraction using a Rigaku MINIFLEX II diffractometer, the X-rays emitted by which are obtained by a copper tube and source (wavelength Kα 1.54 Å).

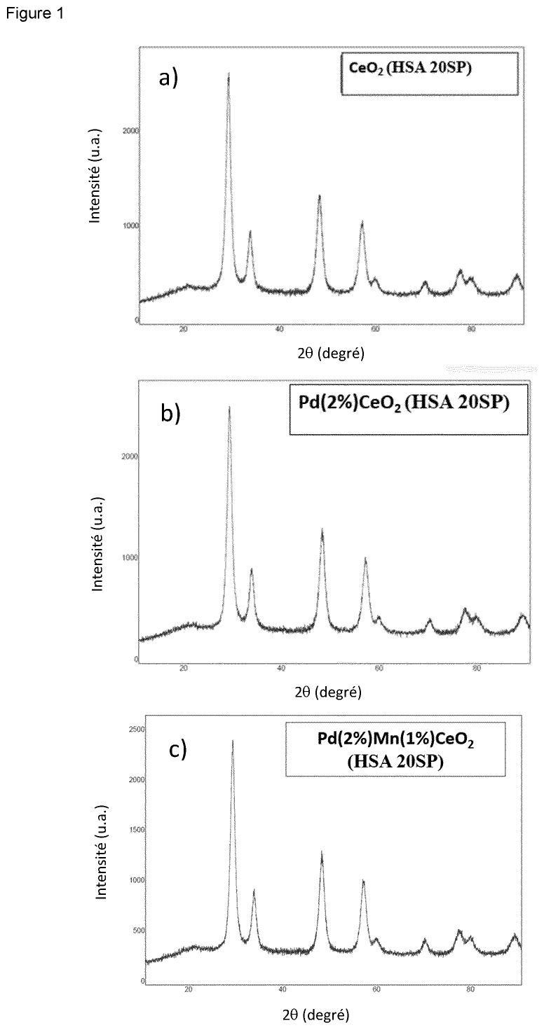

The three diffractograms in FIG. 1 show peaks corresponding to the (111), (200), (220), (311), (222), (400) and (311) planes, attributable to a fluorine structure of cerium oxide (JCPDS 34-0394). There is a distinct broadening of the diffraction peaks.

### Crystallite Size

The size of the crystallites was estimated qualitatively in order to compare with the various catalysts in the prior art, in particular the catalysts and their support described in Kai Li et al. (Front. Chem. Sci. Eng. 2020, 14 (6); 929-936).

The size of the crystallites was evaluated using the following Scherrer formula:

$$\begin{matrix}t = \frac{k . λ}{H . cos θ} & t = crystallite size \\ & k = correction factor = 0.89 \\ & λ = wavelength of the source \\ & H = width at half - height of the peak ( in radians ) \\ & θ = diffraction angle\end{matrix}$$

It should be noted that the correction due to defects in the instrumental optics has not been taken into account in the estimated calculations, as its contribution to peak broadening is considered negligible to a first approximation.

The width at mid-height was estimated using Image J

The crystallite size calculations were carried out using the peak of the diffractograms located at approximately 28 degrees and are reported in the following table:

TABLE 15
Crystallite sizes

| | | Crystallite | Width at |
|---|---|---|---|
| | | sizes | half-height H |
| Reference | Support or Catalyst | (nm) | (degrees) |
| Kai Li et al. | CeO₂ support - | 58.7 | 0.138 |
| | from Alfa Aesar | | |
| Example 1 | CeO₂ support - | 7.9 | 1.02 |
| | HSA 20SP - from Solvay | | |
| Kai Li et al. | Pd/CeO₂ | 42.0 | 0.193 |
| | (calcination treatment at 600 C.) | | |
| Example 3 | Pd(2%)/CeO₂ | 7.7 | 1.048 |
| | (HSA 20 SP) | | |
| Example 5 | Pd(2%)-Mn(1%)/CeO₂ | 7.2 | 1.128 |
| | (HSA 20 SP) | | |

The support used in Kai Li et al. is a commercial cerium oxide powder from Alfa Aesar, with a pore volume of 0.18 mL/g and an average pore size of 12.2 nm with a specific surface area of 5.8 m²/g (BET).

The low value of the sizes, in particular less than 10 nanometres, is an indicator of a low crystallinity structure. The smaller the crystallites, the wider the diffraction peaks. This effect becomes visible for crystallites less than 1 μm in diameter.

The results indicate that the cerium oxide support and the catalysts described by Kai Li et al. have crystallite sizes of 42 nm to 59 nm respectively and the HSA 20SP support and the catalysts according to the invention have crystallite sizes of less than 10 nanometres, i.e. around 8 nm.

In addition to the specific surface area, these results show that the microstructure of the supports and catalysts differs from those described in Kai Li et al. with the catalysts of the invention exhibiting lower crystallinity.

### Example 20: Morphological Characterisation of Catalysts

Morphological analysis was carried out by scanning electron microscopy (SEM) using a ZEISS Sigma HD SEM-FEG apparatus on 3 catalysts of the invention:
the Pd (2%)/CeO₂ catalyst (HSA 85 SP),
and the Pd(2%)/CeO₂ catalyst (HSA 20 SP) prepared according to example 3
and the Pd(2%)—Mn (1%)/CeO₂ catalyst (HSA 20 SP) prepared according to example 5.

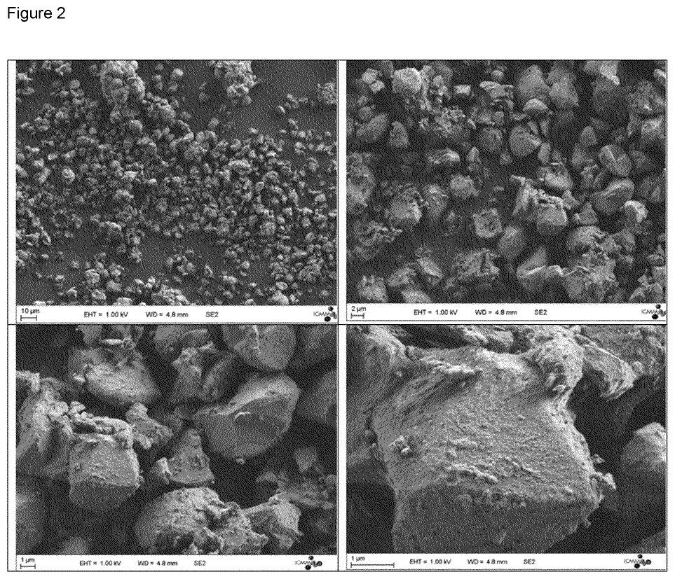

The SEM images shown in FIG. 2 of the Pd (2%)/CeO₂ catalyst (HSA 85 SP) reveal a population made up of particles ranging in size from approximately 1 to around 10 micrometres, of any slightly facetted shape, with a specific surface area of 186 m²/g (BET, see table 2 of example 3).

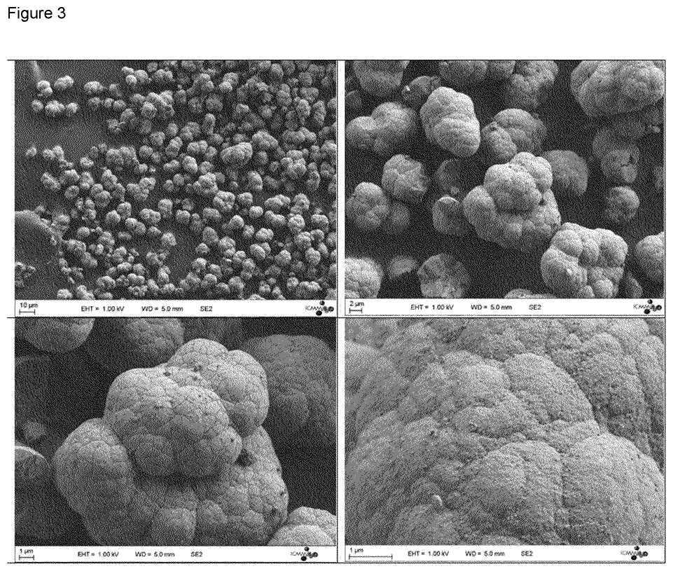

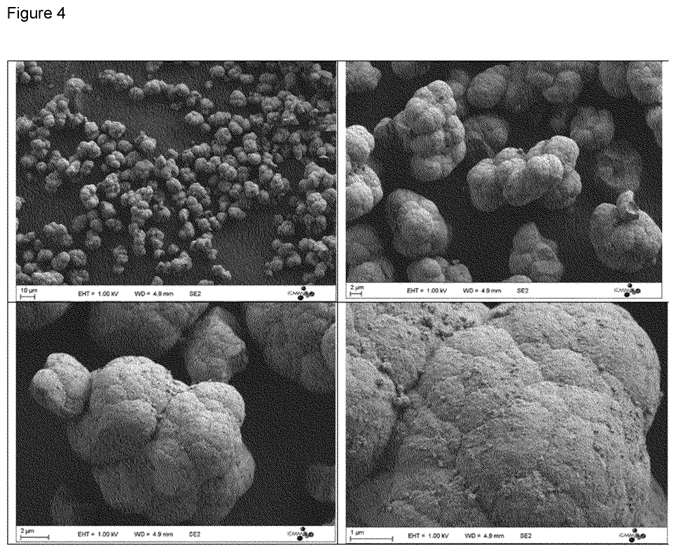

The SEM images of FIG. 3 of the Pd (2%)/CeO₂ catalyst (HSA 20 SP) and FIG. 4 of the Pd (2%)—Mn(1%)/CeO₂ catalyst (HSA 20 SP) are similar because they were prepared using the same HSA 20SP support and differ only in the presence of 1% by weight of manganese. They reveal a homogeneous population made up of particles around ten micrometres in size, in the form of a compacted agglomerate of round particles (like a truffle), with a specific surface area of 142 m²/g (BET, see table 2 of example 3).

SEM images reveal that the morphology of the prepared catalysts differs from the well-crystallized catalysts described in Chao Hu et al. (Catalysis Letter 2002, 152, 503-512) who disclose for Pd/CeO catalysts₂:
a population of cubes around fifty nanometres in size (with a specific surface area of 7.82 m²/g),
an octahedron population of 20 to 80 nm (with a specific surface area of 7.82 m²/g),
a population of rods approximately 9 nm wide and 40 to 140 nm long (with a specific surface area of 66 m²/g)

### Example 21: Analysis of the Surface of Catalysts

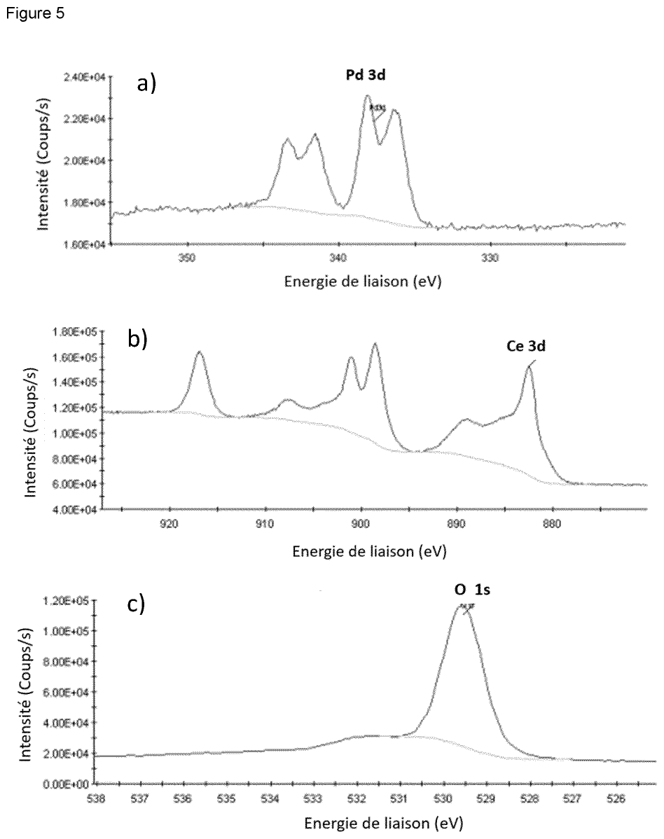

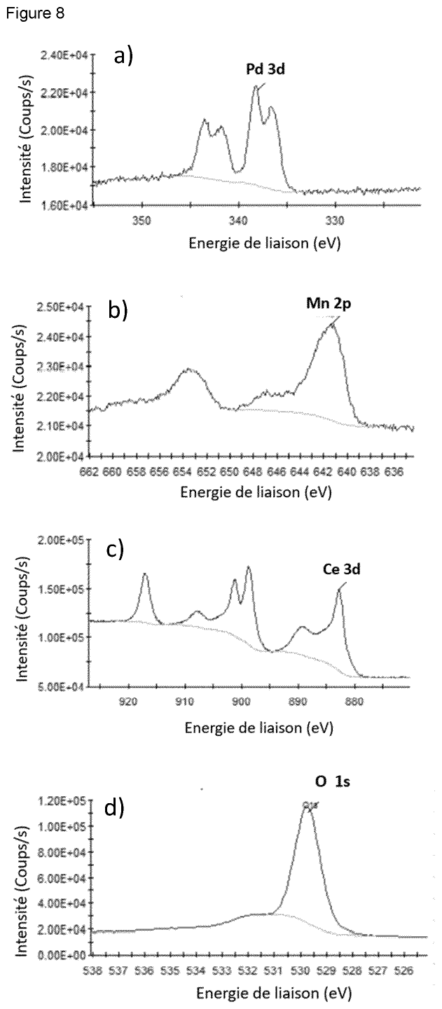

The surface of the Pd (2%)/CeO₂ (HSA 20 SP) catalyst prepared according to example 3 and of the Pd (2%)—Mn(1%)/CeO₂ (HSA 20 SP) catalyst prepared according to example 5 was analysed by XPS. The results are reported in Tables 16 to 18 and FIGS. 5 to 8.

TABLE 16
Binding energy of the Pd(2%)/CeO₂ catalyst (HSA 20 SP)

| Peaks | Binding energy (eV) |
|---|---|
| Pd 3d | 337.71 |
| This 3d | 882.56 |
| O 1s | 529.55 |

TABLE 17
Binding energy of the Pd 3d peaks of
the Pd(2%)/CeO₂ catalyst (HSA 20 SP)

| Peaks | Binding energy (eV) |
|---|---|
| Pd 3d5 PdO | 336.05 |
| Pd 3d3 PdO | 341.36 |
| Pd 3d5 PdₓCe₁₋ₓO₂ | 337.82 |
| Pd 3d3 PdₓCe₁₋ₓO₂ | 343.15 |

TABLE 18
Binding energy of the Pd(2%)-Mn(1%)/CeO₂ catalyst (HSA 20 SP)

| Peaks | Binding energy (eV) |
|---|---|
| Pd 3d | 338.05 |
| Mn 2p3 | 641.42 |
| This 3d | 882.76 |
| O 1s | 529.71 |

TABLE 19
Binding energy of Pd 3d peaks of
Pd(2%)-Mn(1%)/CeO₂ (HSA 20 SP)

| Peaks | Binding energy (eV) |
|---|---|
| Pd 3d5 PdO | 336.11 |
| Pd 3d3 PdO | 341.35 |
| Pd 3d5 PdₓCe₁₋ₓO₂ | 337.88 |
| Pd 3d3 PdₓCe₁₋ₓO₂ | 343.17 |

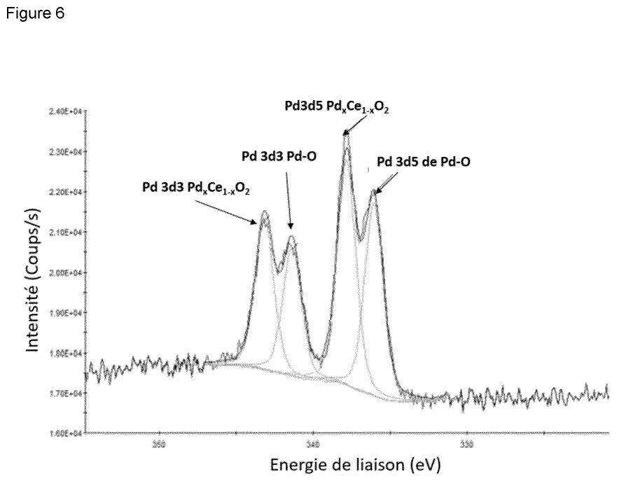

As shown in Table 17 and FIG. 6, the Pd 3d spectrum can be decomposed into 2 contributions, the one at low binding energy (336.1 eV) can be attributed to Pd—O bonds, the other at energies around 337.8 eV can be attributed to the PdₓCe₁₋ₓO₂ species.

The main difference compared with the article D1 Catalysis Letters, 152 (503-512) 2022 is the presence of Pd(II) of oxidation state 2 in the form of palladium oxide PdO instead of Pd(0) of oxidation state (0).

These results show that the surface of the catalysts includes both palladium oxide Pd—O and PdₓCe₁₋ₓO₂ species but does not include palladium metal Pd(0).

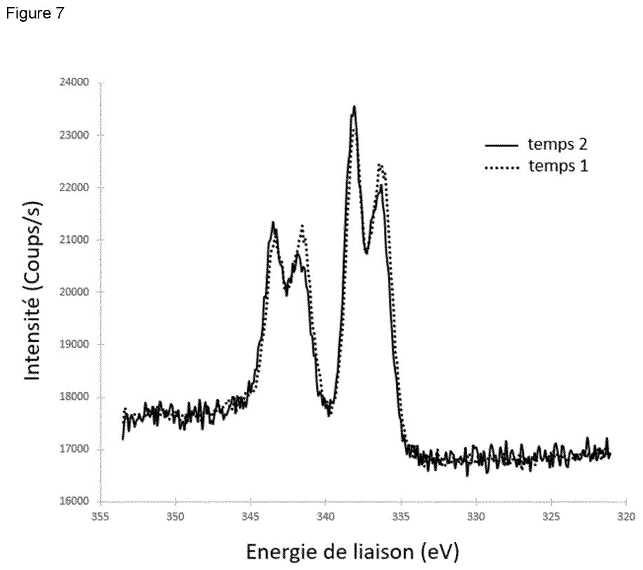

The spectra in FIG. 7 show a variation in the areas of the peak contributions at two different exposure times (time 1 and time 2) and indicate a palladium reduction phenomenon under the X-ray beam, as the ratio of the PdO and PdₓCe₁₋ₓO₂ components varies with time.

1-23. (canceled)

24. A method for selectively preparing an oxalate compound or an oxamide compound comprising a step A of bringing an alcohol or an amine, respectively, into contact with:
carbon monoxide,
an oxidizing agent,
a catalyst of formula Pd—X/CeO₂, wherein X represents an empty set or a dopant element, comprising Pd atoms on a CeO₂ support, in which the catalyst has a surface area, analyzed by BET, from 100 to 250 m/g,²
to obtain a reaction medium which may be pressurized from 0.1 to 15 MPa, and
to obtain the oxalate compound or the oxamide compound.

25. The method for selectively preparing an oxalate compound or an oxamide compound according to claim 24, comprising a step A of bringing an alcohol or an amine, respectively, into contact with a promoter.

26. The method for selectively preparing an oxalate compound or an oxamide compound according to claim 24, comprising a step A of bringing an alcohol or an amine, respectively, into contact with a base.

27. The method for selectively preparing an oxalate compound or an oxamide compound according to claim 24, comprising a step A of bringing an alcohol or an amine, respectively, into contact with a solvent.

28. The method for selectively preparing an oxalate compound or an oxamide compound according to claim 24, further comprising a step B of heating the reaction medium.

29. The method for selectively preparing an oxalate compound or an oxamide compound according to claim 24, wherein the catalyst has a palladium content from 0.1 to 10%, in particular 2%, or 5%, by weight relative to the total weight of the catalyst.

30. The method for selectively preparing an oxalate compound or an oxamide compound according to claim 24, wherein a dopant is chosen from Mn, Mg, Ca, Fe, Ba, Sr, Y, Nb, Zn, Bi, Sn, La, Pr, Nb and Sm, in particular at a content from 0.5 to 10%, preferably 1%, by weight relative to the total weight of the catalyst.

31. The method for selectively preparing an oxalate compound or an oxamide compound according to claim 24, comprising a step A of bringing an alcohol or an amine, respectively, into contact with:
carbon monoxide, in particular used from 0.5 to 8.0 MPa, in particular at 6.5 MPa
oxygen or air, in particular oxygen used from 0.5 to 2.5 MPa, in particular at 1.5 MPa
a promoter, in particular an iodine compound, chosen in particular from tetramethylammonium iodide, potassium iodide or sodium iodide, preferably tetramethylammonium iodide, preferably in a proportion from 0.1 to 5 mol % relative to the alcohol or amine, in particular in a proportion of 0.2 mol %,
a Pd—X/CeO₂ catalyst comprising Pd atoms on a CeO₂ support of the formula Pd—X/CeO₂ wherein X represents the void system or a dopant element, wherein the catalyst has a surface area, analyzed by BET, from 100 to 250 m²/g, preferably the palladium is from 0.01 to 10 mol % relative to the alcohol or amine,
optionally a base, in particular triethylamine, preferably used in a proportion from 0.1 to 5 mol % relative to the alcohol or amine, in particular in a proportion of 0.15 mol %,
optionally a solvent, in particular selected from acetonitrile, tetrahydrofuran, dioxane and toluene, preferably acetonitrile,
to obtain a reaction medium,
optionally a step B of heating the reaction medium,
to obtain the oxalate compound or the oxamide compound.

32. The method for selectively preparing an oxalate compound or an oxamide compound according to claim 24, wherein an oxalate compound is selectively prepared, and wherein the method comprises a step A of bringing an alcohol into contact with:
carbon monoxide,
oxygen or air,
a promoter,
a Pd—X/CeO₂ catalyst, comprising Pd atoms on a CeO₂ support, of the formula Pd—X/CeO₂ wherein X represents the void system or a dopant element, wherein the catalyst has a surface area, analyzed by BET, from 100 to 250 m²/g, preferably in an amount from 0.01 to 10 mol % relative to the alcohol or amine,
to obtain a reaction medium,
a step B of heating said reaction medium, in particular at a temperature from 25 to 200° C., in particular from 60 to 110° C., preferably around 90° C., to obtain the oxalate compound.

33. The method for selectively preparing an oxalate compound or an oxamide compound according to claim 32, wherein the method comprises a step A of bringing an alcohol, into contact with a base.

34. The method for selectively preparing an oxalate compound or an oxamide compound according to claim 32, wherein the method comprises a step A of bringing an alcohol, into contact with a solvent.

35. The method for selectively preparing an oxalate compound or an oxamide compound according to claim 24, wherein an oxalate compound of Formula 2 is selectively prepared, and wherein step A comprises contacting an alcohol of Formula 1: 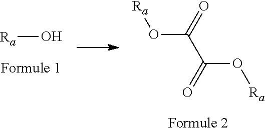
wherein Rₐ represents:
a C₁ to C₂₀ linear or branched alkyl group,
a C₃ to C₁₀ cycloalkyl group,
a C₅ to C₂₀ alkyl-aryl or alkyl-heteroaryl group,
in particular the alcohol is chosen from methanol, ethanol and isopropanol.

36. The method for selectively preparing an oxalate compound or an oxamide compound according to claim 24, wherein an oxamide compound is selectively prepared, and wherein the method comprises a step A of bringing an amine into contact with:
carbon monoxide,
oxygen or air,
a promoter,
a Pd—X/CeO₂ catalyst comprising Pd atoms on a CeO₂ support of the formula Pd—X/CeO₂ wherein X represents the empty set or a dopant element, wherein the catalyst has a surface area, analyzed by BET, from 100 to 250 m²/g, preferably the palladium is from 0.01 to 10 mol % relative to the alcohol or amine,
a solvent,
to obtain a reaction medium comprising the oxamide compound.

37. The method for selectively preparing an oxalate compound or an oxamide compound according to claim 36, wherein an oxamide compound is selectively prepared, and wherein the method comprises a step A of bringing an amine, into contact with a base.

38. The method for selectively preparing an oxalate compound or an oxamide compound according to claim 36, wherein an oxamide compound of Formula 4 is selectively prepared, and wherein step A comprises contacting an amine of Formula 3: 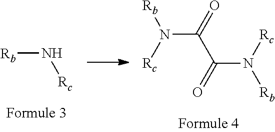
wherein R_{b} and R_{c} independently of one another represent:
a hydrogen atom,
a C₁ to C₂₀ linear or branched alkyl group,
a C₃ to C₁₀ cycloalkyl group,
a C₅ to C₂₀ alkyl-aryl or alkyl-heteroaryl group,
at least one of the groups R_{b} or R_{c} being other than hydrogen,
R_{b} and R_{c} can form a cycle,
in particular the amine is chosen from piperidine, pyrrolidine, butylamine, benzylamine, furfurylamine and cyclohexylamine.

39. A palladium/cerium dioxide catalyst, comprising palladium on a cerium dioxide support, of the formula Pd—X/CeO₂, wherein X represents the void or a doping element,
wherein the catalyst has a surface area, analyzed by BET, from 100 to 250 m²/g, in particular from 100 and 200 m²/g,
and wherein the catalyst has a palladium content from 0.1 to 10%, in particular 2% or 5%, by weight relative to the total weight of the catalyst,
optionally the dopant being selected from the following group of elements: Mn, Mg, Ca, Fe, Ba, Sr, Y, Nb, Zn, Bi, Sn, La, Pr, Nb and Sm, preferably the dopant being Mn, in particular the said catalyst comprising a dopant content from 0.5 and 10%, in particular 1%, by weight relative to the total weight of the catalyst.

40. The catalyst according to claim 39, wherein the catalyst has a fluorine-type structure by XRD and a crystallite size of less than 30 nanometers according to the Scherrer formula, in particular less than 20 nanometers, preferably less than 10 nanometers, or
wherein the catalyst has a crystallinity level from 0 to 50%, preferably from 0 to 20%.

41. The catalyst according to claim 39, wherein said catalyst is in the form of particles of average micrometric size, in particular from 1 to 100 μm.

42. The catalyst according to claim 39, wherein the surface of the said catalyst, analyzed by XPS, comprises from 90 to 100% palladium in the oxidation state (II), in particular in the form of Pd—O or a PdₓCe₁₋ₓO₂ solid solution, x varying from 0.01 to 1.

43. A method for preparing a Pd—X/CeO₂ catalyst according to claim 39, wherein the method comprises:
a step D of impregnating a palladium salt, in particular palladium nitrate, on a cerium dioxide support, to obtain a homogeneous material,
in particular said step D comprises:
the use of a support with a surface area from 100 to 300 m²/g, in particular from 150 to 160 m²/g or from 270 to 280 m²/g,
and using a palladium salt concentration calculated to obtain a palladium content of 0.1 to 10%, by weight relative to the total weight of the catalyst,
optionally using another salt selected from precursors of dopants from the following group of elements: Mn, Mg, Ca, Fe, Ba, Sr, Y, Nb, Zn, Bi, Sn, La, Pr, Nb and Sm, in particular the manganese salt, the dopant salt concentration is calculated to obtain a dopant content from 0.5 to 10% by weight relative to the total weight of the catalyst,
a step E for drying the material, in particular at a temperature from 60 to 100° C., in particular 80° C., preferably for a period from 10 to 24 hours, in particular 16 hours, and
a step F of palladium activation, in particular comprising calcination at a temperature from 200 to 1000° C., in particular 600° C., preferably for a period from 1 to 15 hours, in particular 2 hours, to obtain said catalyst.
